(19) United States
(12) Patent Application Publication
(10) Pub. No.: US 2009/0111860 A1
(43) Pub. Date: Apr. 30, 2009

(54) SULFONAMIDE COMPOUNDS USEFUL AS ADG RECEPTOR MODULATORS

(75) Inventors: Gurmit Grewal, Waltham, MA (US); Edward Hennessy, Waltham, MA (US); Victor Kamhi, Waltham, MA (US); Danyang Li, Waltham, MA (US); Vibha Oza, Waltham, MA (US); Jamal Carlos Saeh, Waltham, MA (US); Qibin Su, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/297,791

(22) PCT Filed: Apr. 20, 2007
(86) PCT No.: PCT/GB2007/001427

§ 371 Date: Oct. 20, 2008

Related U.S. Application Data
(60) Provisional application No. 60/745,295, filed on Apr. 21, 2006.

(51) Int. Cl. A61K 31/425; C07D 275/02; A61P 35/00; C07D 231/12; C07D 261/08; A61K 31/42; A61K 31/415

(52) U.S. Cl. 514/372; 548/214; 514/406; 548/375.1; 548/247; 514/378

(57) ABSTRACT

The present invention relates to compounds of formula (I) that mediate Edg, including Edg-1, processes for their preparation, pharmaceutical compositions containing them as the active ingredient, to their use as medicaments and to their use in the manufacture of medicaments for use in the treatment in warm-blooded animals such as humans of diseases that have a significant vascularization or inflammatory component such as in tumor-related diseases. The present invention also relates to compounds that inhibit a5bl, and also that exhibit appropriate selectivity profile(s) against other integrins.

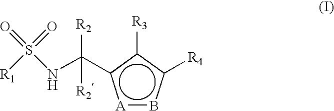

### BACKGROUND OF THE INVENTION

EDG (endothelial differentiation gene) receptors belong to a family of closely related, lipid activated G-protein coupled receptors. EDG-1, EDG-3, EDG-5, EDG-6, and EDG-8 (also known as S1P1, S1P3, S1P2, S1P4, and S1P5) are identified as receptors specific for sphingosine-1-phosphate (S1P). EDG2, EDG4, and EDG7 (known also as LPA1, LPA2, and LPA3, respectively) are receptors specific for lysophosphatidic (LPA). Among the S1P receptor isotypes, EDG-1, EDG-3 and EDG-5 are widely expressed in various tissues, whereas the expression of EDG-6 is confined largely to lymphoid tissues and platelets, and that of EDG-8 to the central nervous system.

EDG receptors are responsible for signal transduction and are thought to play an important role in cell processes involving cell development, proliferation, maintenance, migration, differentiation, plasticity and apoptosis. Certain EDG receptors are associated with diseases mediated by the de novo or deregulated formation of vessels—for example, for diseases caused by ocular neovascularisation, especially retinopathies (diabetic retinopathy, age-related macular degeneration); psoriasis; hemangiomas such as “strawberry-marks”; various inflammatory diseases, such as arthritis, especially rheumatoid arthritis, arterial atherosclerosis and atherosclerosis occurring after transplants, endometriosis or chronic asthma; and tumor diseases; or by lymphocyte interactions, for example, in transplantation rejection, autoimmune diseases, inflammatory diseases, infectious diseases and cancer. An alteration in EDG receptor activity contributes to the pathology and/or symptomology of these diseases. Accordingly, molecules that themselves alter the activity of EDG receptors are useful as therapeutic agents in the treatment of such diseases.

### SUMMARY OF THE INVENTION

These and other needs are met by the present invention which is directed to a compound of formula I

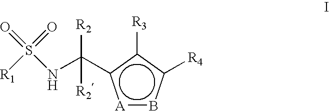

in free or pharmaceutically acceptable salt form, wherein:

A and B are each independently N, NRₐ, O, S, or CR_{b};

Rₐ is H, (C₁-C₆)alkyl, C(O)—(C₁-C₆)alkyl, C(O)—NR′R″, CO₂(C₁-C₆)alkyl;

R_{b}H, halo, (C₁-C₆)alkyl, cyano, —C(O)—(C₁-C₆)alkyl, —CO₂(C₁-C₆)alkyl, C(O)—NR′R″, wherein R′ and R″ are each independently at each occurrence H or (C₁-C₆)alkyl or X—R_{c}; —CO₂H, —SO₂NHR;

R₁ is aryl, heteroaryl, (C₁-C₆)alkyl, aralkyl, heterocycloalkyl, or heteroaralkyl;

R₂ and R_{2′} are each independently H, (C₁-C₆)alkyl, aryl, heteroaryl, aralkyl, or heteroaralkyl, or taken together with the carbon to which they are attached form C═O;

R₃ and R₄ are each independently H, halo, (C₁-C₆)alkyl, (C₃-C₆)cycloalkyl, (C₃-C₆)cycloalkyl(C₁-C₆)alkyl, heterocycloalkyl, aralkyl, aryl, (C₂-C₆)alkenyl, (C₂-C₆)alkynyl, or heteroaralkyl, or X—R_{c};

X is S, O, or NR_{d};

R_{c} is H or (C₁-C₆)alkyl;

R_{d} is H, (C₁-C₆)alkyl, aryl, heteroaryl, heterocyclo, (C₂-C₆)alkenyl, (C₂-C₆)alkynyl, aralkyl, heteroaralkyl, (C₃-C₆)cycloalkyl(C₁-C₆)alkyl, heterocycloalkyl(C₁-C₆)alkyl, acyl, acyloxy, acylamino, or (C₁-C₆)alkoxycarbonyl(C₁-C₆)alkyl, or cyano; and

each R₁, R₂, R_{2′}, R₃, Rₐ, R_{b}, R_{c}, and R_{d} may be optionally substituted on carbon by azido, halo, nitro, cyano, hydroxy, trifluoromethoxy, NR′R″, —CO₂H, C(O)—(C₁-C₆)alkyl, —CO₂(C₁-C₆)alkyl, —C(O)—NR′R″, S(C₁-C₆), SOₚ(C₁-C₆)alkyl, SOₚNH(C₁-C₆)alkyl, SOₚNR′R″ (C₂-C₆)alkenyl, (C₂-C₆)alkynyl, or (C₁-C₆)alkoxy, wherein R′ and R″ are each independently hydrogen, (C₁-C₆)alkyl, (C₃-C₆)cycloalkyl, (C₃-C₆)cycloalkyl(C₁-C₆)alkyl, or aryl.

The invention further provides a compound of formula II

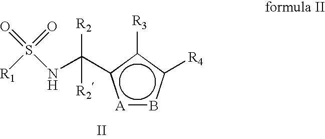

or a pharmaceutically acceptable salt thereof, wherein:

A and B are each independently N, NRₐ, O, S, or CR_{b};

Rₐ is H, (C₁-C₆)alkyl, C(O)—(C₁-C₆)alkyl, C(O)—NR′R″, CO₂(C₁-C₆)alkyl;

R_{b}H, halo, (C₁-C₆)alkyl, cyano, —C(O)—(C₁-C₆)alkyl, —CO₂(C₁-C₆)alkyl, C(O)—NR′R″, wherein R′ and R″ are each independently at each occurrence H or (C₁-C₆)alkyl or X—R_{c}; —CO₂H, —SO₂NHR;

R₁ is optionally substituted aryl, heteroaryl, (C₁-C₆)alkyl, aralkyl, heterocycloalkyl, or heteroaralkyl;

R₂ and R_{2′} are each independently H, (C₁-C₆)alkyl, aryl, heteroaryl, aralkyl, or heteroaralkyl, or taken together with the carbon to which they are attached form C═O;

R₃ and R₄ are each independently (C₁-C₆)alkyl, (C₃-C₆)cycloalkyl(C₁-C₆)alkyl, heterocycloalkyl, aralkyl, (C₂-C₆)alkenyl, (C₂-C₆)alkynyl, or heteroaralkyl, or X—R_{c};

X is S, O, or NR_{d};

R_{c} is H or (C₁-C₆)alkyl;

R_{d} is H, (C₁-C₆)alkyl, aryl, heteroaryl, heterocyclo, (C₂-C₆)alkenyl, (C₂-C₆)alkynyl, aralkyl, heteroaralkyl, (C₃-C₆)cycloalkyl(C₁-C₆)alkyl, heterocycloalkyl(C₁-C₆)alkyl, acyl, acyloxy, acylamino, or (C₁-C₆)alkoxycarbonyl(C₁-C₆)alkyl, or cyano; and

each R₁, R₂, R_{2′}, R₃, Rₐ, R_{b}, R_{c}, and R_{d} may be optionally substituted on carbon by azido, halo, nitro, cyano, hydroxy, trifluoromethoxy, NR′R″, —CO₂H, C(O)—(C₁-C₆)alkyl, CO₂(C₁-C₆)alkyl, —C(O)—NR′R″, S(C₁-C₆), SOₚ(C₁-C₆)alkyl, SOₚNH(C₁-C₆)alkyl, SOₚNR′R″ (C₂-C₆)alkenyl, (C₂-C₆)alkynyl, or (C₁-C₆)alkoxy, wherein R′ and R″ are each independently hydrogen, (C₁-C₆)alkyl, (C₃-C₆)cycloalkyl, (C₃-C₆)cycloalkyl(C₁-C₆)alkyl, or aryl.

The invention is also directed to a compound III, which is selected from a group consisting of:

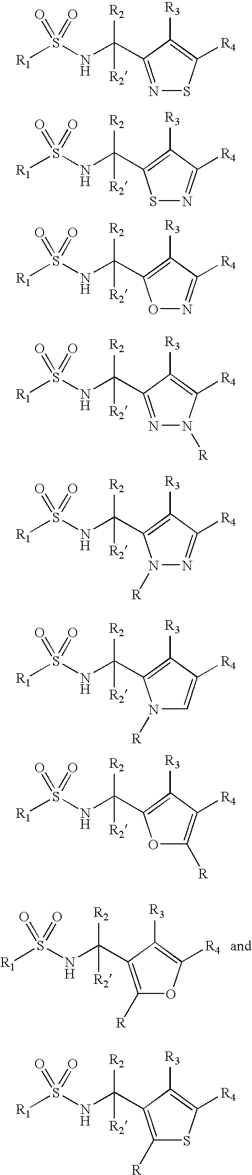

or a pharmaceutically acceptable salt thereof, wherein

R is H, (C₁-C₆)alkyl, C(O)—(C₁-C₆)alkyl, C(O)—NR′R″ or CO₂(C₁-C₆)alkyl and R₁, R₂, R_{2′}, R₃, and R₄ are as defined for a compound of formula I.

The invention further provides a compound of formulas I, II or III, in free or salt form as follows:
1.1 Compounds of formulas I, II or III, wherein A and B are each independently N, NRₐ, O, S, or CR_{b} wherein Rₐ and R_{b} are hereinbefore described.
1.2 Compounds of formulas I, II or III or 1.1, wherein one of A or B is N and the other is NRₐ.
1.3 Compounds of formulas I, II or III, 1.1 or 1.2, wherein A is N and B is NRₐ, wherein Rₐ is hereinbefore described.
1.4 Compounds of formulas I, II or III or any of 1.1-1.3, wherein A is N and B is NRₐ, wherein Rₐ is (C₁-C₆)alkyl.
1.5 Compounds of formulas I, II or III or any of 1.1-1.4, wherein A is N and B is NRₐ, wherein Rₐ is methyl.
1.6 Compounds of formulas I, II or III or any of 1.1-1.3, wherein A is N and B is NRₐ, wherein Rₐ is H.
1.7 Compounds of formulas I, II or III or 1.1, wherein one of A or B is N and the other is O.
1.8 Compounds of formulas I, II or III, 1.1 or 1.7, wherein A is N and B is O.
1.9 Compounds of formulas I, II or III or 1.1, wherein one of A or B is N and the other is S.
1.10 Compounds of formulas I, II or III, 1.1 or 1.9, wherein A is N and B is S.
1.11 Compounds of formulas I, II or III or 1.1, wherein one of A or B is N and the other is CR_{b}, wherein R_{b} is hereinbefore described.
1.12 Compounds of formulas I, II or III, 1.1 or 1.11, wherein A is N and B is R_{b}, wherein R_{b} is hereinbefore described.
1.13 Compounds of formulas I, II or III or any of 1.1-1.12, wherein R₁ is optionally substituted aryl, heteroaryl, (C₁-C₆)alkyl, aralkyl, heterocycloalkyl, or heteroaralkyl.
1.14 Compounds of formulas I, II or III or any of 1.1-1.13, wherein R₁ is optionally substituted aryl (e.g., phenyl).
1.15 Compounds of formulas I, II or III or any of 1.1-1.14, wherein R₁ is halo substituted aryl (e.g., chlorophenyl).
1.16 Compounds of formulas I or II or any of 1.1-1.15, wherein R₁ is 4-chlorophen-1-yl.
1.17 Compounds of formulas I, II or III or any of 1.1-1.16, wherein R₂ and R_{2′} are each independently selected from a group consisting of H, (C₁-C₆)alkyl, aryl, heteroaryl, aralkyl, and heteroaralkyl, or R₂ and R_{2′} taken together with the carbon to which they are attached form C═O.
1.18 Compounds of formulas I, II or III or any of 1.1-1.17, wherein R₂ and R_{2′} are independently H, (C₁-C₆)alkyl or aralkyl (e.g., phenyl(C₁-C₆)alkyl).
1.19 Compounds of formulas I, II or III or any of 1.1-1.18, wherein R₂ and R_{2′} are independently H, methyl or benzyl.
1.20 Compounds of formulas I, II or III or any of 1.1-1.19, wherein one of R₂ or R_{2′} is methyl and the other is H.
1.21 Compounds of formulas I, II or III or any of 1.1-1.19, wherein one of R₂ or R_{2′} is benzyl and the other is H.
1.22 Compounds of formulas I, II or III or any of 1.1-1.21, wherein R₃ and R₄ are each independently selected from a group consisting of (C₁-C₆)alkyl, (C₃-C₆)cycloalkyl(C₁-C₆)alkyl, heterocycloalkyl, aralkyl, (C₂-C₆)alkenyl, (C₂-C₆)alkynyl, heteroaralkyl and X—R_{c} wherein X and R_{c} are hereinbefore described.
1.23 Compounds of formulas I, II or III or any of 1.1-1.22, wherein R₃ is selected from a group consisting of (C₁-C₆)alkyl, (C₃-C₆)cycloalkyl(C₁-C₆)alkyl, heterocycloalkyl, aralkyl, (C₂-C₆)alkenyl, (C₂-C₆)alkynyl, or heteroaralkyl, or X—R_{c} wherein X and R_{c} are hereinbefore described.
1.24 Compounds of formulas I, II or III or any of 1.1-1.23, wherein R₃ is (C₁-C₆)alkyl.
1.25 Compounds of formulas I, II or III or any of 1.1-1.24, wherein R₃ is ethyl.
1.26 Compounds of formulas I, II or III or any of 1.1-1.23, wherein R₃ is (C₂-C₆)alkynyl.
1.27 Compounds of formulas I, II or III or any of 1.1-1.23 or 1.26, wherein R₃ is ethynyl.
1.28 Compounds of formulas I, II or III or any of 1.1-1.27, wherein R₄ is selected from a group consisting of (C₁-C₆)alkyl, (C₃-C₆)cycloalkyl(C₁-C₆)alkyl, heterocycloalkyl, aralkyl, (C₂-C₆)alkenyl, (C₂-C₆)alkynyl, or heteroaralkyl, or X—R_{c} wherein X and R_{c} are hereinbefore described.
1.29 Compounds of formulas I, II or III or any of 1.1-1.28, wherein R₄ is (C₁-C₆)alkyl.
1.30 Compounds of formulas I, II or III or any of 1.1-1.29, wherein R₄ is methyl.
1.31 Compounds of formulas I, II or III or any of 1.1-1.29, wherein R₄ is ethyl.
1.32 Compounds of formulas I, II or III or any of 1.1-1.28, wherein R₄ is X—R_{c}, and X and R_{c} are hereinbefore described.
1.33 Compounds of formulas I, II or III, any of 1.1-1.28 or 1.32, wherein R₄ is X—R_{c}, wherein X is O and R_{c} is (C₁-C₆)alkyl.
1.34 Compounds of formulas I, II or III, any of 1.1-1.28 or 1.32-1.33, wherein R₄ is methoxy.
1.35 Compounds of formulas I, II or III, any of 1.1-1.28 or 1.32, wherein R₄ is X—R_{c}, wherein X is NR_{d} and R_{d} and R_{c} are hereinbefore described.
1.36 Compounds of formulas I, II or III, any of 1.1-1.28, 1.32 or 1.35, wherein R₄ is NH₂.
1.37 Compounds of formula I or any of 1.1-1.21, wherein R₃ and R₄ are each independently selected from a group consisting of H, halo, (C₃-C₆)cycloalkyl or aryl.
1.38 Compounds of formula I, any of 1.1-1.21 or 1.37, wherein R₃ is halo (e.g., chloro, bromo or iodo).
1.39 Compounds of formula I, any of 1.1-1.27 or 1.37-1.38, wherein R₄ is aryl.
1.40 Compounds of formula I, any of 1.1-1.27 or 1.37-1.39, wherein R₄ is phenyl.
1.41 Compounds of formula I, any of 1.1-1.27 or 1.37-1.38, wherein R₄ is (C₃-C₆)cycloalkyl.
1.42 Compounds of formula I, any of 1.1-1.27, 1.37-1.38 or 1.41, wherein R₄ is cyclopropyl.
1.43 Compounds of formulas I, II or III or any of 1.1-1.42 selected from any of the following:

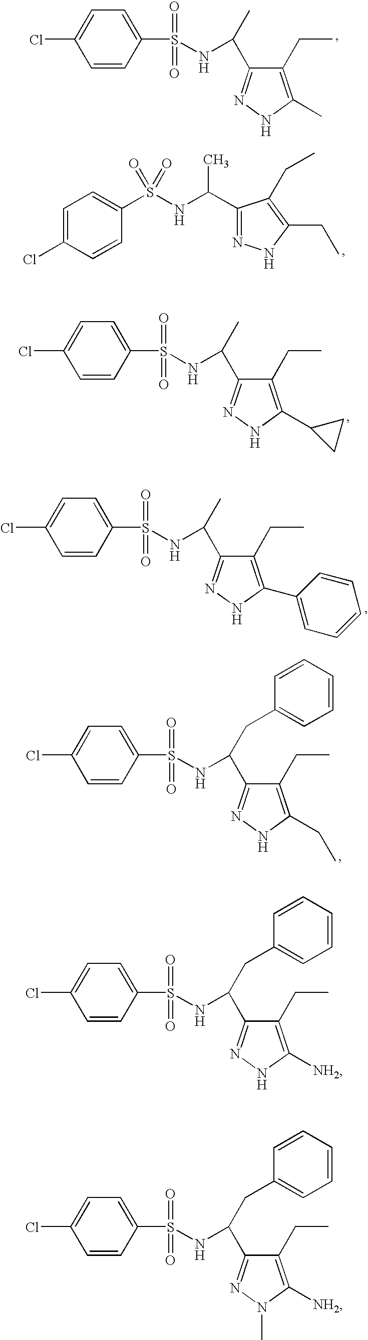

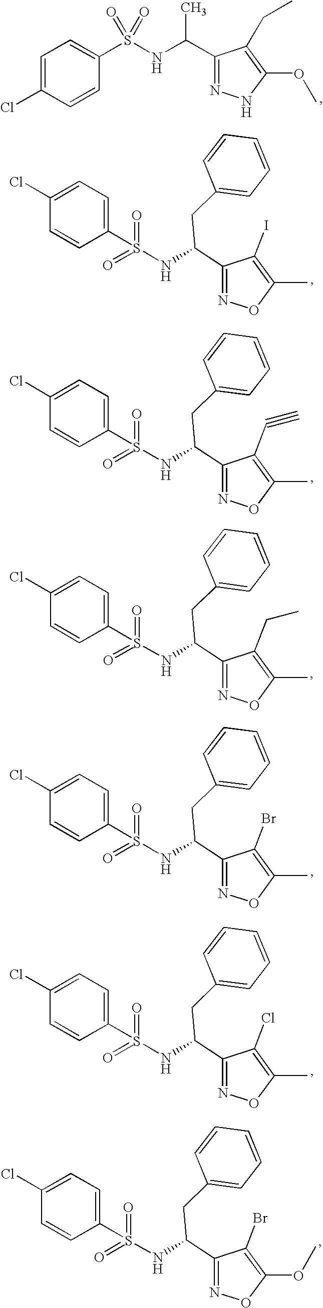

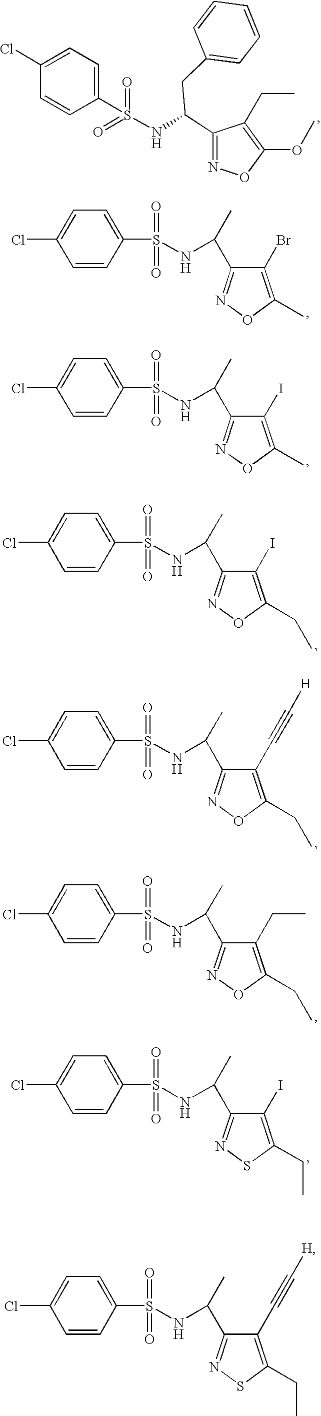

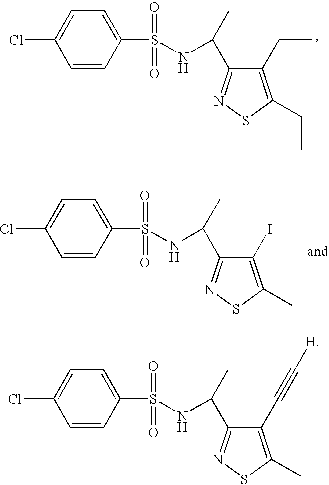

The present invention also provides for compounds of formula I or II in free or pharmaceutically acceptable salt form, wherein:

A is N;

B is NRₐ, O or S;

Rₐ is H or (C₁-C₆)alkyl;

R₁ is aryl;

R₂ and R_{2′} are each independently H, (C₁-C₆)alkyl, or aralkyl;

R₃ and R₄ are each independently halo, (C₁-C₆)alkyl, (C₃-C₆)cycloalkyl, aryl, (C₂-C₆)alkynyl, or X—R_{c};

X is O or NR_{d};

R_{c} is H or (C₁-C₆)alkyl;

R_{d} is H; and

each R₁, R₂, R_{2′}, R₃, Rₐ, and R_{c} may be optionally substituted on carbon by azido, halo, nitro, cyano, hydroxy, trifluoromethoxy, NR′R″, —CO₂H, C(O)—(C₁-C₆)alkyl, —CO₂(C₁-C₆)alkyl, —C(O)—NR′R″, S(C₁-C₆), SOₚ(C₁-C₆)alkyl, SOₚNH(C₁-C₆)alkyl, SOₚNR′R″ (C₂-C₆)alkenyl, (C₂-C₆)alkynyl, or (C₁-C₆)alkoxy, wherein R′ and R″ are each independently hydrogen, (C₁-C₆)alkyl, (C₃-C₆)cycloalkyl, (C₃-C₆)cycloalkyl(C₁-C₆)alkyl, or aryl.

The present invention further provides compounds of formula I or II in free or pharmaceutically acceptable salt form, wherein:

A is N;

B is NRₐ;

Rₐ is H or (C₁-C₆)alkyl;

R₁ is phenyl;

One of R₂ and R_{2′} is H and the other is (C₁-C₆)alkyl or aralkyl;

R₃ and R₄ are each independently halo, (C₁-C₆)alkyl, (C₃-C₆)cycloalkyl, aryl, (C₂-C₆)alkynyl, or X—R_{c};

X is O or NR_{d};

R_{c} is H or (C₁-C₆)alkyl;

R_{d} is H; and

each R₁, R₂, R_{2′}, R₃, Rₐ, and R_{c} may be optionally substituted on carbon by halo.

The present invention also provides for compounds of formula I or II in free or pharmaceutically acceptable salt form, wherein:

A is N;

B is O or S;

R₁ is phenyl;

R₂ and R_{2′} are each independently H, (C₁-C₆)alkyl, or aralkyl;

R₃ and R₄ are each independently halo, (C₁-C₆)alkyl, (C₃-C₆)cycloalkyl, aryl, (C₂-C₆)alkynyl, or X—R_{c};

X is O or NR_{d};

R_{c} is H or (C₁-C₆)alkyl;

R_{d} is H; and

each R₁, R₂, R_{2′}, R₃, and R_{c} may be optionally substituted on carbon by halo.

What is also provided is a compound of formulas I, II or III or any of 1.1-1.43 or a pharmaceutically acceptable salt, prodrug, or solvate thereof in association with a pharmaceutically acceptable carrier, diluent, or excipient.

What is also provided is a compound of formulas I, II or III or any of 1.1-1.43 or a pharmaceutically acceptable salt, prodrug, or solvate thereof, useful for controlling pathologically angiogenic diseases, thrombosis, cardiac infarction, coronary heart diseases, arteriosclerosis, tumors, osteoporosis, inflammations or infections.

What is also provided is a method (Method I) of treating a disease or condition selected from a group consisting of pathologically angiogenic diseases, thrombosis, cardiac infarction, coronary heart diseases, arteriosclorosis, tumors, osteoporosis, inflammations and infections, which method comprises administering to a patient in need of such treatment a compound of formula I, II or III or any of 1.1-1.43 or a pharmaceutically acceptable salt, prodrug, or solvate thereof.

What is also provided is a compound of formulas I, II or III or any of 1.1-1.43 in free or pharmaceutically acceptable salt, prodrug, or solvate thereof, which is an Edg-1 antagonist useful for controlling pathologically angiogenic diseases, thrombosis, cardiac infarction, coronary heart diseases, arteriosclerosis, tumors, osteoporosis, inflammations or infections.

What is also provided is a method (Method II) of treating a disease or condition mediated by Edg-1 which comprises administering to a patient in need of such treatment a compound of formulas I, II or III or any of 1.1-1.43 or a pharmaceutically acceptable salt, prodrug, or solvate thereof; for example wherein the disease or condition mediated by Edg-1 is selected from (i) diseases mediated by the de novo or deregulated formation of vessels—for example, for diseases caused by ocular neovascularisation, especially retinopathies (diabetic retinopathy, age-related macular degeneration); psoriasis; hemangiomas such as “strawberry-marks”; (ii) various inflammatory diseases, such as arthritis, especially rheumatoid arthritis, arterial atherosclerosis and atherosclerosis occurring after transplants, endometriosis or chronic asthma; (iii) tumor diseases; and (iv) by lymphocyte interactions, for example, in transplantation rejection, autoimmune diseases, inflammatory diseases, infectious diseases and cancer.

What is also provided is a compound of formulas I, II or III or any of 1.1-1.43, in free or pharmaceutically acceptable salt, prodrug or solvate form, for use as a medicament.

What is also provided is a use of a compound of formulas I, II or III or any of 1.1-1.43, in free or pharmaceutically acceptable salt, prodrug or solvate form, in the manufacture of a medicament for use in Method I or II.

What is also provided is a compound of formulas I, II or III or any of 1.1-1.43, in free or pharmaceutically acceptable salt, prodrug or solvate form for use in Method I or II.

What is also provided is a pharmaceutical composition comprising a compound of formulas I, II or III or any of 1.1-1.43, in free or pharmaceutically acceptable salt, prodrug or solvate form, in association with a pharmaceutically acceptable excipient or carrier for use in Method I or II.

What is also provided is a process (Process I) for the preparation of a compound of formula I, II or II or any of 1.1-1.43, in free or pharmaceutically acceptable salt, prodrug or solvate form as summarized in Scheme 1 infra.

What is also provided is a process (Process II) for the preparation of a compound of formula I, II or II or any of 1.1-1.43, in free or pharmaceutically acceptable salt, prodrug or solvate form, which process comprises the step of treating:
a) a compound of formula A:

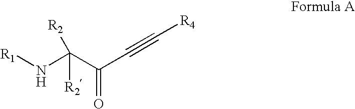

wherein Rₐ, R₁, R₂, R_{2′}, and R₄ are hereinbefore described;
b) with (i) NH₂OH; (ii) Rₐ—NHNH₂; or (iii) hydroxylamine-O-sulfonic acid and sodium hydrogen sulfide.

In one embodiment, Process II further comprises the step of (i) halogenating the compound obtained from step (b) of Process II to obtain the compound the present invention wherein R₃ is halo; or (ii) alkylating the compound obtained from step (i) to recover the compound of the present invention wherein R₃ is alkynyl.

In another embodiment, the invention also provides a process (Process III) for the preparation of a compound of formula I, II or II or any of 1.1-1.43, in free or pharmaceutically acceptable salt, prodrug or solvate form, which process comprises the step of treating:

a) a compound of formula B or C:

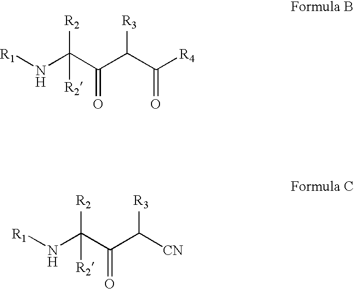

wherein Rₐ, R₁, R₂, R_{2′}, R₃ and R₄ are hereinbefore described;

b) with Rₐ—NHNH₂.

In another embodiment, the invention also provides a process (Process IV) for the preparation of a compound of formula I, II or II or any of 1.1-1.43, wherein R₄ is OH or C₁-₆alkoxy, in free or pharmaceutically acceptable salt, prodrug or solvate form, which process comprises the step of treating:

a) a compound of formula D:

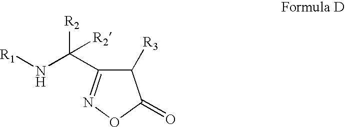

wherein Rₐ, R₁, R₂, R_{2′} and R₃ are hereinbefore described;

b) with trimethylsilylmethyl diazane.

In yet another embodiment, the invention also provides a process (Process V) for the preparation of a compound of formula I, II or II or any of 1.1-1.43, wherein R₄ is OH or C₁₋₆alkoxy, in free or pharmaceutically acceptable salt, prodrug or solvate form, which process comprises the step of treating:
a) a compound of formula E:

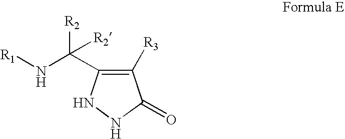

b) with (i) a base (e.g., cesium carbonate, potassium carbonate, sodium carbonate) and (ii) haloC₁₋₆alkyl (e.g., iodomethyl) wherein Rₐ, R₁, R₂, R_{2′} and R₃ are hereinbefore described.

In yet another embodiment, the invention also provides a process (Process VI) for the preparation of a compound of formula I, II or II or any of 1.1-1.43, wherein R₄ is OH or C₁₋₆alkoxy, in free or pharmaceutically acceptable salt, prodrug or solvate form, which process comprises the step of treating:

a) a compound of formula F:

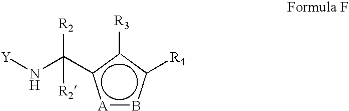

wherein Y is H or a leaving group (e.g., tert-butoxycarbonyl);

b) with R₁—X wherein X is halo (e.g., iodomethane); and

c) a base.

### DETAILED DESCRIPTION OF THE INVENTION

Unless otherwise stated, the following terms used in the specification and claims have the following meanings.

### DEFINITIONS

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, pentyl, and the like.

“Alkylene” means a linear saturated divalent hydrocarbon radical of one to six carbon atoms or a branched saturated divalent hydrocarbon radical of three to six carbon atoms, e.g., methylene, ethylene, propylene, 2-methylpropylene, pentylene, and the like.

“Alkenyl” means a linear monovalent hydrocarbon radical of two to six carbon atoms or a branched monovalent hydrocarbon radical of three to six carbon atoms, containing at least one double bond, e.g., ethenyl, propenyl, and the like.

“Alkynyl” means an alkyl group having one or more carbon-carbon triple bonds, e.g., ethynyl.

“Cycloalkyl” means a saturated monovalent cyclic hydrocarbon radical of three to six ring carbons, e.g., cyclopropyl, cyclohexyl, and the like.

“Aryl” means a monovalent monocyclic or bicyclic aromatic hydrocarbon radical of 6 to 10 ring atoms, and optionally substituted independently with one or more substituents, preferably one, two or three substituents selected from alkyl, haloalkyl, heteroalkyl, cycloalkyl, cycloalkylalkyl, halo, cyano, nitro, acyloxy, alkoxy, optionally substituted phenyl, heteroaryl, heteroaralkyl, amino, monosubstituted amino, disubstituted amino, acylamino, hydroxylamino, amidino, guanidino, cyanoguanidinyl, hydrazino, hydrazido, —OR [where R is hydrogen, alkyl, haloalkyl, alkenyl, cycloalkyl, cycloalkylalkyl, optionally substituted phenyl, heteroaryl or heteroaralkyl], —S(O)ₙR [where n is an integer from 0 to 2 and R is hydrogen, alkyl, haloalkyl, alkenyl, cycloalkyl, cycloalkylalkyl, optionally substituted phenyl, heteroaryl, heteroaralkyl, amino, mono or disubstituted amino], —NRSO₂R′ (where R is hydrogen or alkyl and R′ is alkyl, amino, monosubstituted or disubstituted amino) —C(O)R (where R is hydrogen, alkyl, alkenyl, cycloalkyl, heteroalkyl, haloalkyl or optionally substituted phenyl), —COOR (where R is hydrogen, alkyl, optionally substituted phenyl, heteroaryl or heteroaralkyl), -(alkylene)-COOR (where R is hydrogen, alkyl, optionally substituted phenyl, heteroaryl or heteroaralkyl), methylenedioxy, 1,2-ethylenedioxy, —CONR′R″ or -(alkylene)CONR′R″ (where R′ and R″ are independently selected from hydrogen, alkyl, cycloalkyl, haloalkyl, cycloalkylalkyl, optionally substituted phenyl, heteroaryl and heteroaralkyl). More specifically the term aryl includes, but is not limited to, phenyl, 1-naphthyl, 2-naphthyl, and derivatives thereof.

“Aralkyl” means a radical —Rₐ—R_{b} where Rₐ is bound to R_{b} and Rₐ is an alkylene group and R_{b} is an aryl group as defined above e.g., benzyl, and the like.

“Heterocycle” or “heterocyclyl” means a saturated or partially unsaturated cyclic radical of 3 to 8 ring atoms in which one or two ring atoms are heteroatoms selected from NH, NRₐ as defined above, O, SO, OR SO₂.

“Heteroaryl” means an optionally substituted monovalent monocyclic radical of 5 or 6 ring atoms containing one, two, or three ring heteroatoms selected from N, O, or S, the remaining ring atoms being C. The term heteroaryl includes, but is not limited to pyridyl, pyrrolyl, thiophene, pyrazolyl, thiazolyl, imidazolyl, pyrimidinyl, thiadiazolyl, carbazolyl, and derivatives thereof.

“Heteroaralkyl” means a radical —Rₐ—R_{b} where Rₐ is bound to R_{b} and Rₐ is an alkylene group and R_{b} is a heteroaryl group as defined above e.g., pyridin-3-ylmethyl, 3-(benzofuran-2-yl)propyl, and the like.

“Optionally substituted” means that the group at issue is optionally substituted independently with one, two or three substituents selected from alkyl, haloalkyl, halo, nitro, cyano, —OR (where R is hydrogen or alkyl), —NRR′ (where R and R′ are independently of each other hydrogen or alkyl), —COOR (where R is hydrogen or alkyl) or —CONR′R″ (where R′ and R″ are independently selected from hydrogen or alkyl), or as otherwise provided.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula I may have chiral centres and/or geometric isomeric centres (E- and Z-isomers), and it is to be understood that the invention encompasses all such optical, diastereoisomers and geometric isomers that possess EDG inhibitory activity. The invention further relates to any and all tautomeric forms of the compounds of the formula I that possess CSF-1R kinase inhibitory activity.

It is also to be understood that certain compounds of the formula I can exist in solvated as well as unsolvated forms such as, for example, hydrated forms. It is to be understood that the invention encompasses all such solvated forms that possess EDG kinase inhibitory activity.

The term “Edg-1 mediated” disease or condition herein refers to any disease or condition associated with, caused by, affected by, triggered by or involving the EDG-1 receptor. Such diseases or conditions include, but not limited to pathologically angiogenic diseases, thrombosis, cardiac infarction, coronary heart diseases, arteriosclerosis, tumors, osteoporosis, inflammations and infections.

In the description of the synthetic methods described herein, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. Therefore, at times, reaction may require to be run at elevated temperature or for a longer or shorter period of time. It is also understood by one skilled in the art of organic synthesis that functionality present on various portions of the molecule must be compatible with the reagents and reactions proposed. If not commercially available, starting materials for these processes may be made by procedures, which are selected from the chemical art using techniques similar or analogous to the synthesis of known compounds. All references cited herein are hereby incorporated in their entirety by reference.

The term “halogenation” herein refers to the introduction of an halogen radical onto an organic compound either by substitution or addition. Halogenation is typically done treating the compound with, for example, bromine, chlorine or iodine. Alternatively, halogenation may also be achieved by using, for example, N-bromosuccinimide or N-chlorosuccinimide.

The term “alkylation” herein refers to the introduction of an alkyl radical onto an organic compound by substitution or addition. As used in the present invention, the term encompasses the addition of an acetylide (e.g., ethynyl(trimethyl)silane) to an aryl halide (e.g., isoxazole) to recover ethynyl derivative of the compound of the present invention. Generally, copper (I) halide, palladium and/or Tetrakis(triphenylphosphine)palladium(0) (Pd(PPh₃)₄) is required.

The term “base” herein refers to carbonate, bicarbonate, phosphate or hydroxide of an alkali or alkaline earth metal (e.g. sodium, magnesium, calcium, potassium, cesium or barium); or organic bases such as amine bases (e.g., triethylamine, diisopropylethylamine, trimethylamine, etc.).

As used in the process of preparing the compounds of the present invention, RₐNHNH₂ may be in anhydrous or hydrate form (e.g., monohydrate).

### Preparation of Invention Compounds

Compounds of the invention can be prepared as provided in Scheme 1. The skilled artisan will recognize that Scheme 1 can be adopted readily for the synthesis of invention compounds from starting sulfonamide starting materials other than the one depicted. The skilled artisan will recognize that the invention compounds can be prepared from chiral starting materials or via racemic synthesis, followed by chiral separation, to isolate the enantiomers.

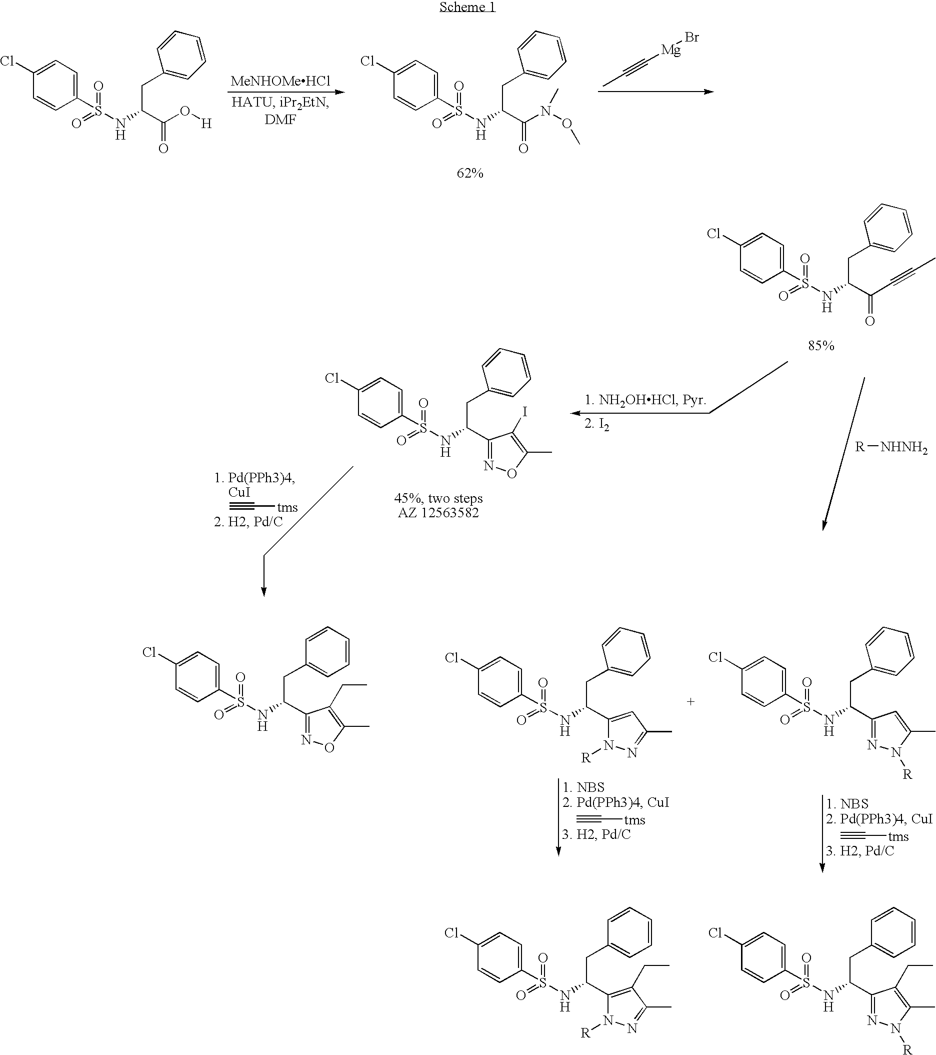

### Pharmaceutical Formulations

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of infection is an amount sufficient to symptomatically relieve in a warm-blooded animal, particularly a human the symptoms of infection, to slow the progression of infection, or to reduce in patients with symptoms of infection the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds of the present invention are capable of forming salts with various inorganic and organic acids and bases and such salts are also within the scope of this invention. Examples of such acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Base salts include ammonium salts, alkali metal salts such as sodium, lithium and potassium salts, alkaline earth metal salts such as aluminum, calcium and magnesium salts, salts with organic bases such as dicyclohexylamine salts, N-methyl-D-glucamine, and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. Non-toxic physiologically-acceptable salts are preferred, although other salts are also useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

In order to use a compound of the formula I, II or III or any of 1.1-1.43 or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

### Combinations

The anti-cancer treatment defined herein may be applied as a sole therapy or may involve, in addition to the compound of the invention, conventional surgery or radiotherapy or chemotherapy. Such chemotherapy may include one or more of the following categories of anti-tumour agents:
1. antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
2. cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
3. agents which inhibit cancer cell invasion (for example metalloproteinase inhibitors like marimastat and inhibitors of urokinase plasminogen activator receptor function);
4. inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
5. antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
6. vascular damaging agents such as Combretastatin A4 and compounds disclosed in International Patent Applications WO 99/02166, WO 00/40529, WO 00/41669, WO 01/92224, WO 02/04434 and WO 02/08213;
7. antisense therapies, for example those which are directed to the targets listed above, such as ISIS 2503, an anti-ras antisense;
8. gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
9. immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

### Biological Activity

The following assay can be used to measure the effects of the compounds of the present invention as S1P1/Edg1 inhibitors.

### A. In Vitro Cell Based Receptor Activation Assay-Transfluor Assay

This cell-based assay was designed to assess the ability of small molecule antagonists to inhibit activation of the GPCR S1P1 in the presence of its cognate ligand S1P. The assay used technology initially developed by Norak Biosciences (Xsira Pharmaceutical) and presently owned by Molecular Devices. A human osteogenic sarcoma (U2OS) cell line overexpressing the EDG-1/S1P1) receptor as well as a beta-arrestin/green fluorescent protein (GFP) construct hereafter termed EDG-1 Transfluor U2OS WT Clone #37 was employed.

Using a high content screening approach (Cellomics Arrayscan), receptor activity was measured by assessment of the relocalization of beta-arrestin GFP in response to stimulation of EDG-1 by S1P. Specifically, EDG-1 Transfluor U2OS WT Clone #37 cells were plated at a density of 6250 cells in 40 uL medium per well in 384 well plastic bottomed microtiter plates (BD Falcon) and incubated overnight at 37° C./5% CO₂. Prior to screening, compounds were dissolved in 100% dimethyl sulfoxide (DMSO) to a final stock concentration of 10 mM. Compounds were then serially diluted at 30× final concentration in EDG-1 Transfluor cell growth medium containing 30% DMSO using the Tecan Genesis instrument. These 30× plates were then diluted to 6× final concentration with EDG-1 Transfluor growth medium just prior to dosing. Cells were then dosed with 10 uL per well of 6× compound dilutions or 6% DMSO and pre-incubated for 15 minutes at room temperature. Cell plates were dosed with 10 uL per well 6× S1P EDG-1 Transfluor growth medium, then incubated for 45 minutes at 37° C./5% CO₂. Final concentration in the well of DMSO was 1%, compound was 1× (3-fold, 9 point IC50 dilutions starting at 100 uM final concentration), and either 375 nM or 750 nM S1P ligand. Cell plates were then fixed by adding 50 uL per well of 5% formaldehyde in 1× Dulbecco's phosphate buffered saline (DPBS) directly and incubating for 30 minutes at room temperature in darkness. Fixative was removed and replaced with 50 uL per well of 1× DPBS, after which cells were stained with 10 ug/mL final concentration of Hoechst 33342 (Molecular Probes) for 15 minutes at room temperature in darkness. Stain was then removed from the plates and replaced with 50 uL per well of 1× DPBS using the BioTek ExL405 plate washer. Plates were then sealed and analysed on the Cellomics Arrayscan using the GPCR signalling algorithm. EC₅₀ values were then calculated using IDBS ActivityBase software.

In this assay, compounds of the invention exhibit EC₅₀ values <100 μM; i.e., the compound of example 1 had an EC₅₀ of 0.68 uM.

### EXPERIMENTAL SECTION

The invention will now be illustrated in the following Examples in which, generally:

(i) operations were carried out at ambient temperature, i.e. in the range 17 to 25° C. and under an atmosphere of an inert gas such as nitrogen or argon unless otherwise stated;

(ii) in general, the course of reactions was followed by thin layer chromatography (TLC) and/or analytical high pressure liquid chromatography (HPLC); the reaction times that are given are not necessarily the minimum attainable;

(iii) when necessary, organic solutions were dried over anhydrous magnesium sulphate, work-up procedures were carried out using traditional layer separating techniques or an ALLEXIS (MTM) automated liquid handler, evaporations were carried out either by rotary evaporation in vacuo or in a Genevac HT-4/EZ-2.

(iv) yields, where present, are not necessarily the maximum attainable, and when necessary, reactions were repeated if a larger amount of the reaction product was required;

(v) in general, the structures of the end-products of the Formula I were confirmed by nuclear magnetic resonance (NMR) and/or mass spectral techniques; electrospray mass spectral data were obtained using a Waters ZMD or Waters ZQ LC/mass spectrometer acquiring both positive and negative ion data, generally, only ions relating to the parent structure are reported; proton NMR chemical shift values were measured on the delta scale using either a Bruker Spectrospin DPX300 spectrometer operating at a field strength of 300 MHz, a Bruker Dpx400 operating at 400 MHz or a Bruker Advance operating at 500 MHz.

The following abbreviations have been used: s, singlet; d, doublet; t, triplet; q, quartet; m, multiplet; br, broad;

(vi) unless stated otherwise compounds containing an asymmetric carbon and/or sulphur atom were not resolved;

(vii) intermediates were not necessarily fully purified but their structures and purity were assessed by TLC, analytical HPLC, infra-red (IR) and/or NMR analysis;

(viii) unless otherwise stated, column chromatography (by the flash procedure) and medium pressure liquid chromatography (MPLC) were performed on Merck Kieselgel silica (Art. 9385);

(ix) preparative HPLC was performed on C18 reversed-phase silica, for example on a Waters ‘Xterra’ preparative reversed-phase column (5 microns silica, 19 mm diameter, 100 mm length) using decreasingly polar mixtures as eluent, for example decreasingly polar mixtures of water (containing 1% acetic acid or 1% aqueous ammonium hydroxide (d=0.88)) and acetonitrile;

(x) the following analytical HPLC methods were used; in general, reversed-phase silica was used with a flow rate of about 1 ml per minute and detection was by Electrospray Mass Spectrometry and by UV absorbance at a wavelength of 254 nm; for each method Solvent A was water and Solvent B was acetonitrile; the following columns and solvent mixtures were used:—

Preparative HPLC was performed on C18 reversed-phase silica, on a Phenominex “Gemini” preparative reversed-phase column (5 microns silica, 110A, 21.1 mm diameter, 100 mm length) using decreasingly polar mixtures as eluent, for example decreasingly polar mixtures of water (containing 0.1% formic acid or 0.1% ammonia) as solvent A and acetonitrile as solvent B; either of the following preparative HPLC methods were used:

Method A: a solvent gradient over 9.5 minutes, at 25 mls per minute, from a 85:15 mixture of solvents A and B respectively to a 5:95 mixture of solvents A and B.

Method B: a solvent gradient over 9.5 minutes, at 25 mls per minute, from a 60:40 mixture of solvents A and B respectively to a 5:95 mixture of solvents A and B.

(xi) where certain compounds were obtained as an acid-addition salt, for example a mono-hydrochloride salt or a di-hydrochloride salt, the stoichiometry of the salt was based on the number and nature of the basic groups in the compound, the exact stoichiometry of the salt was generally not determined, for example by means of elemental analysis data;

(xii) the following abbreviations have been used:—
DMF N,N-dimethylformamide
DMSO dimethylsulphoxide
THF tetrahydrofuran
DMA N-dimethylacetamide
DCM Dichloromethane
HATU O-(7-Azabenzotriazol-1-Yl)-N,N,N′,N′-Tetramethyluronium Hexafluoro-Phosphate
TBAF Tetra-n-butylammonium fluoride
   The compounds of the present invention as exemplified in Table 1 were synthesized as described below:

TABLE 1

| Ex | Compound | ¹H NMR | M/Z | Int |
|---|---|---|---|---|
| 1 | 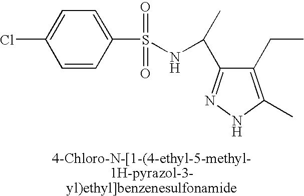 | (400 MHz, DMSO-D6) δ ppm0.82-0.92 (m, 3H) 1.23 (d, 3H)1.99 (s, 3H) 2.10-2.21 (m, 2H)4.30-4.39 (m, 1H) 7.53 (m,2H) 7.61-7.70 (m, 2H) 7.99(d, 1H) 11.99 (s, 1H) | | 1 |
| 2 | 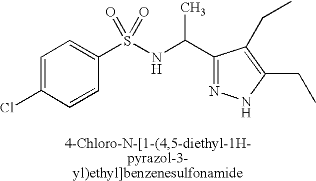 | (400 MHz, DMSO-D6) δ ppm0.89 (t, J = 7.58 Hz, 3H) 1.06 (t,J = 7.58 Hz, 3H) 1.24 (d, J = 7.07 Hz,3H) 2.11-2.22 (m, 2H)2.38 (q, J = 7.58 Hz, 2H) 4.33-4.44 (m, 1H) 7.49-7.59 (m, 2H)7.63-7.65 (m, 2H) 8.02 (m,1H),12.01 (broad s, 1H). | 341 | 1 |
| 3 | 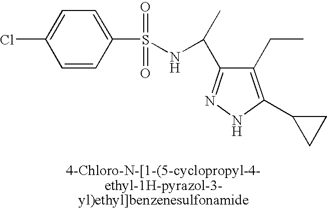 | (400 MHz, DMSO-D6) δ ppm0.54-0.65 (m, 2H) 0.75-0.85(m, 2H) 0.93 (t, 3H) 1.24 (d, 3H)1.62 (s, 1H) 2.19-2.30 (m, 2H)4.31-4.41 (m, 1H) 7.51 (m,2H) 7.63 (m, 2H) 8.02 (d, 1H)11.81 (s, 1H) | | 1 |
| 4 | 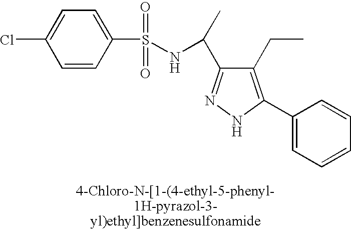 | (400 MHz, DMSO-D6) δ ppm0.93 (t, J = Hz, 3H) 1.33 (d,J = 6.82 Hz, 3H) 2.36 (m, 2H)4.48 (m, 1H) 7.34-7.46 (m, 5H)7.51 (m, 2H) 7.67 (m, 2H)8.16 (d, J = 8.08 Hz, 1H) 12.60(broad s, 1H) | 389 | 1 |
| 5 | 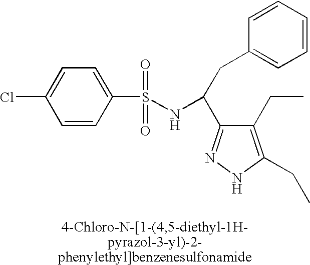 | (400 MHz, DMSO-D6) δ ppm0.64 (t, J = 7.45 Hz, 3H) 1.02 (t,J = 7.58 Hz, 3H) 1.95 (m, 2H)2.25-2.35 (m, 2H) 2.99 (m, 2H) 4.39 (m, 1H) 7.00 (m, 2H)7.08-7.17 (m, 3H) 7.40 (m, 2H)7.46-7.56 (m, 2H) 8.24 (m,1H). | 417 | 2 |
| 6 | 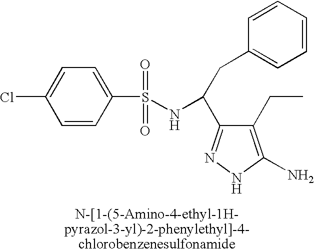 | (400 MHz, DMSO-D6) δ ppm0.58 (t, J = 7.33 Hz, 3H) 1.81-1.92 (m, 2H) 2.87 (m, 1H) 2.98(m, 1H) 4.19-4.30 (m, 3H)6.96 (m, 2H) 7.06-7.16 (m, 3H)7.46-7.52 (m, 2H) 7.60 (m,2H) 8.11 (m, 1H) 10.98 (broads, 1H) | 404 | 3 |
| 7 | 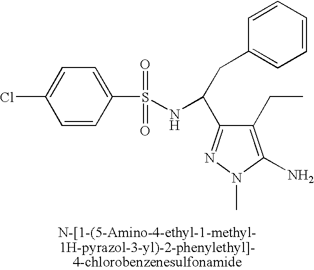 | (400 MHz, DMSO-D6) δ ppm0.64 (t, J = Hz, 3H) 1.94 (m,2H) 2.76-2.88 (m, 1H) 2.95(m, 1H) 4.26 (m, 1H) 4.60-4.71 (m, 2H) 6.99-7.05 (m, 2H)7.08-7.18 (m, 3H) 7.38-7.47 (m, 2H) 7.47-7.56 (m, 2H)8.09 (d, J = 8.34 Hz, 1H) | 418 | 4 |
| 8 | 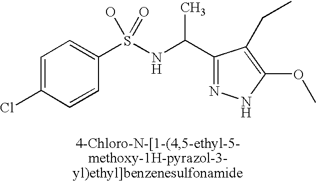 | (400 MHz, DMSO-D6) δ ppm0.85 (m, 3H) 1.26 (m, 2H) 1.98-2.09 (m, 2H) 3.69 s, 3H) 4.24-4.35 (m, 1H) 7.56 (m, 2H)7.65 (m, 2H) 8.04-8.15 (m, 1H)11.26 (s, 1H) | 343 | 5 |
| 9 | 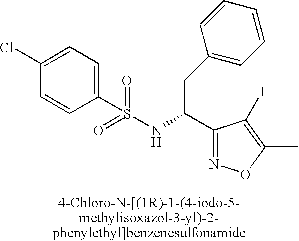 | (300 MHz,CDCl₃) δ 7.56 (2H, m), 7.23 (5H,m), 7.03 (2H, m), 5.26 (1H, d),4.68 (1H, m), 3.10 (2H, m), 2.32(3H, s) | 502 | 6 |
| 10 | 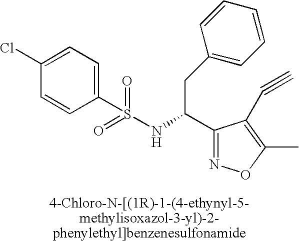 | (300 MHz,CDCl₃) δ 7.56 (2H, m), 7.28(2H, d), 7.18 (3H, m), 7.00 (2H,m), 5.29 (1H, br), 4.81 (1H, m),3.28 (1H, s), 3.14 (2H, d), 2.31(3H, s) | 400 | 6 |
| 11 | 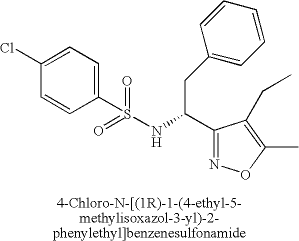 | (300 MHz,CDCl₃) δ 7.56 (2H, m), 7.28(2H, d), 7.18 (3H, m), 7.00 (2H,m), 5.29 (1H, d), 4.56 (1H, m),3.08 (2H, d), 2.21 (3H, s), 1.97(2H, m), 0.79 (3H, t) | 404 | 6 |
| 12 | 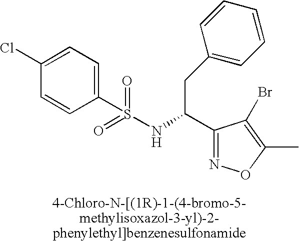 | (300 MHz, CDCl₃)δ 7.58 (2H, m), 7.28 (5H, m),7.06 (2H, m), 5.23 (1H, d), 4.78(1H, m), 3.14 (2H, m), 2.30 (3H,s). | 454 | 7 |
| 13 | 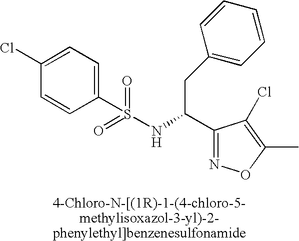 | (300 MHz, CDCl₃)δ 7.50 (2H, m), 7.17 (5H, m),6.96 (2H, m), 5.18 (1H, d), 4.70(1H, m), 3.05 (2H, m), 2.20 (3H,s) | 410 | 7 |
| 14 | 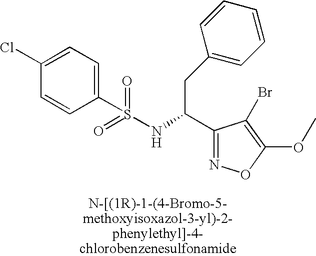 | (300 MHz, CDCl₃)7.62 (2H, m),7.28 (5H, m), 7.08 (2H, m), 5.24(1H, d), 4.70 (1H, m), 4.09 (3H,s), 3.13 (2H, m) | 470 | 8 |
| 15 | 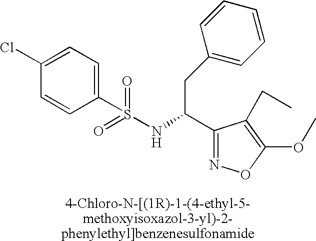 | (300 MHz, CDCl₃)δ 7.52 (2H, δ), 7.27 (2H, δ), 7.15(3H, m), 6.97 (2H, m), 4.47 (2H,m), 3.93 (3H, s), 3.01 (2H, dd),1.84 (2H, q), 0.77 (3H, t) | 420 | 8 |
| 16 | 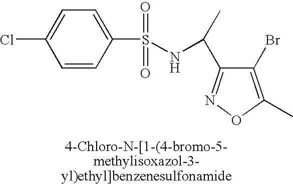 | (CDCl3): δ 7.66 (d, 2H), 7.25(d, 2H), 5.01 (d, 1H), 4.69 (m,1H), 2.00 (s, 3H), 1.46 (d, 3H) | 379.66 | 9 |
| 17 | 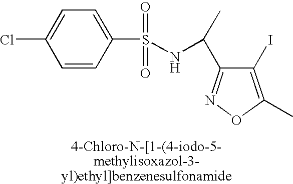 | (CDCl3): δ 7.70 (d, 2H), 7.35 (d,2H), 5.15 (d, 1H), 4.80 (m, 1H),2.20 (s, 3H), 1.59 (d, 3H) | 426.65 | 9 |
| 18 | 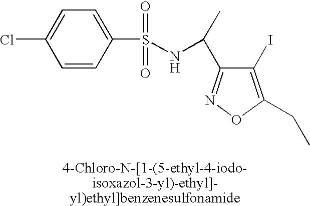 | (300 MHz, CDCl3-D) δ ppm1.08-1.14 (t, 3H) 1.43-1.48 (d, 3H)2.33-2.42 (qm, 2H) 4.62-4.73 (m, 1H) 5.26 (d, 1H) 7.24(dd, 2H) 7.60 (dd, 2H) | 441 | 10 |
| 19 | 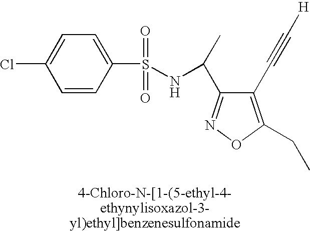 | (300 MHz, CDCl3-D) δ ppm1.17-1.23 (t, 3H) 1.5-1.57 (d, 3H)2.4-2.57 (q, 2H) 3.30 (s, 1H)4.78-4.88 (m, 1H) 5.16-5.22 (d,1H) 7.31 (dd, 2H) 7.65 (dd, 2H) | 339 | 10 |
| 20 | 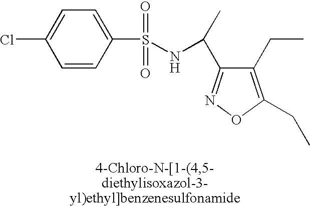 | (300 MHz, CDCl3-D) δ ppm0.92 (t, 3H) 1.15 (t, 3H) 1.42-1.45 (d, 2H) 2.31-2.45 (m, 4H)4.54-4.65 M, 1H), 5.03-5.10 (d,1H) 7.27 (dd, 2H) 7.52 (dd, 2H) | 343 | 10 |
| 21 | 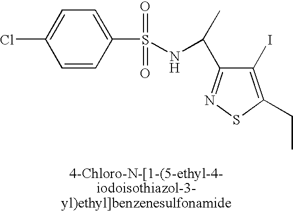 | (300 MHz, CDCl₃) δ 1.29 (t, 3H) 1.46 (d, 3H) 2.75 (q, 2H)4.76 (m, 1H) 7.27 (d, 2H) 7.63(d, 2H) | 457 | 10 |
| 22 | 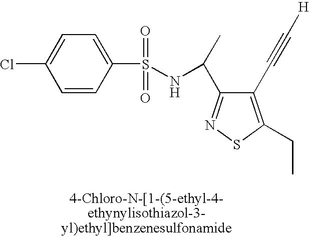 | (300 MHz, CDCl₃) δ 1.29 (t, 3H)1.52 (d, 3H) 2.84 (q, 2H) 3.47(s, 1H) 4.81 (m, 1H) 5.67 (d, 1H)7.28 (d, 2H) 7.63 (d, 2H) | 355 | 10 |
| 23 | 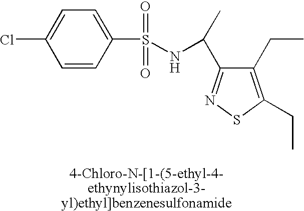 | (300 MHz, CDCl₃) δ 1.04 (t, 3H)1.31 (t, 3H) 1.51 (d, 3H)2.43 (m, 2H) 2.77 (q, 2H) 4.65(m, 1H) 6.18 (d, 1H) 7.30 (d, 2H) 7.64 (d, 2H) | 359 | 10 |
| 24 | 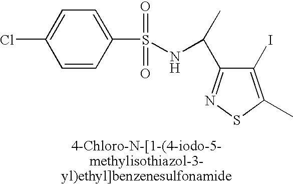 | (CDCl3) δ 7.55 (d, 2H), 7.21 (d,2H), 5.65 (d, 1H), 4.67 (m, 1H),2.30 (s, 3H), 1.34 (d, 3H) | 442.7 | 11 |
| 25 | 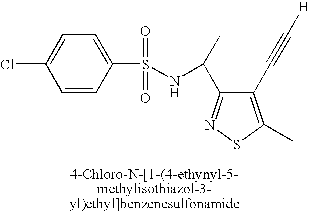 | (CDCl3) δ 7.65 (d, 2H), 7.28 (d,2H), 5.60 (d, 1H), 4.75 (m, 1H),3.35 (s, 1H), 2.41 (s, 3H), 1.50(d, 3H) | 340.85 | 11 |

General method for the preparation of Examples 1-4 from intermediate 1 as represented below for example 2:

### 4-Chloro-N-[1-(4,5-diethyl-1H-pyrazol-3-yl)ethyl]benzenesulfonamide

### Example 2

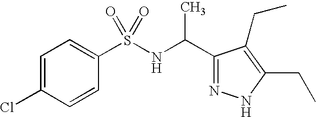

A test tube equipped with a stir bar is charged with 4-chloro-N-(1-methyl-2-oxopentyl)benzenesulfonamide (Intermediate 1, 162 mg, 0.561 mmol) and is evacuated and backfilled with N₂. Anhydrous toluene (2.0 mL) is added, and the resulting solution is cooled to 0° C. A solution of LiHMDS (1.0 M in THF; 2.0 mL, 2.0 mmol) is added in one portion, and the resulting mixture is allowed to stir at 0° C. for 2-3 min. Propionyl chloride (70 μL, 0.81 mmol) is then added in one portion, and the mixture is allowed to stir at 0° C. for 2 min and is allowed to warm to room temperature over 3 min. Glacial HOAc (0.50 mL) is added to quench the reaction, followed by absolute EtOH (2 mL). Hydrazine monohydrate (150 μL, 3.1 mmol) is added, and the mixture is allowed to stir at room temperature. After 45 min, the reaction is partitioned between EtOAc and H₂O. The aqueous layer is extracted with EtOAc, and the combined organics are washed with brine, dried (MgSO₄), filtered, and concentrated. The crude material is purified by silica gel chromatography (gradient elution; R_{f} in 50:50 hexanes:EtOAc=0.23) to give a viscous oil that is lyophilized to give a colorless solid (54 mg, 28%).

Example 5 may be prepared in two steps from intermediate 2a as outlined below:

### 4-Chloro-N-[1-(4,5-diethyl-1H-pyrazol-3-yl)-2-phenylethyl]benzenesulfonamide

### Example 5

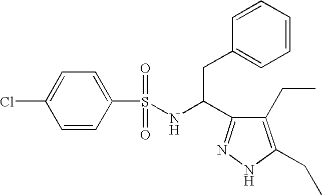

A 25 mL round bottom flask is charged with N-(1-benzyl-3-ethyl-2,4-dioxohexyl)-4-chlorobenzenesulfonamide (Intermediate 2a, 104 mg, 0.25 mmol) and MeOH (4.0 mL). Hydrazine monohydrate (50 μL, 1.03 mmol) is added, and the solution is allowed to stir at room temperature for 1 h. The volatile components are removed under reduced pressure, and the crude material is purified by silica gel chromatography (EtOAc as eluent) to give a colorless oil. Lyophilization affords a solid material (16 mg, 15%).

### N-[1-(5-Amino-4-ethyl-1H-pyrazol-3-yl)-2-phenylethyl]-4-chlorobenzenesulfonamide

### Example 6

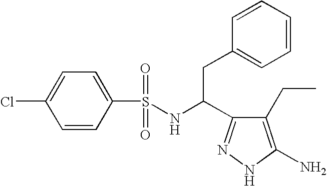

A 50 mL round bottom flask is charged with N-(1-{5-amino-1-[(4-chlorophenyl)sulfonyl]-4-ethyl-1H-pyrazol-3-yl}-2-phenylethyl)-4-chlorobenzenesulfonamide (Intermediate 3a, 203 mg, 0.35 mmol) and dioxane (2 mL). A solution of NaOH (94 mg, 2.35 mmol) in H₂O (1 mL) is added, and the mixture is heated to 50° C. After 3 h, the reaction is treated with saturated NH₄Cl (3 mL) and is extracted (2×) with CH₂Cl₂. The combined organics are washed with brine, dried (MgSO₄), filtered, and concentrated. The product is crystallized from MeOH/H₂O to give a pale yellow solid (89 mg, 63%).

### N-[1-(5-amino-4-ethyl-1-methyl-1H-pyrazol-3-yl)-2-phenylethyl]-4-chlorobenzene-sulfonamide

### Example 7

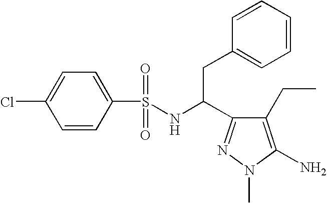

A 25 mL round bottom flask is charged with tert-butyl[1-(5-amino-4-ethyl-1-methyl-1H-pyrazol-3-yl)-2-phenylethyl]carbamate (Intermediate 4, 108 mg, 0.31 mmol) and 4 N HCl/dioxane (2 mL). The resulting solution is allowed to stir at room temperature for 1 h and the volatile components are then removed under reduced pressure. The residue is treated with CH₂Cl₂ (2 mL) and triethylamine (220 μL, 1.6 mmol), followed by 4-chlorobenzenesulfonyl chloride (84 mg, 0.40 mmol). The mixture is allowed to stir at room temperature for 90 min and then the mixture is partitioned between CH₂Cl₂ and H₂O. The aqueous layer is further extracted with CH₂Cl₂, and the combined organics are washed with H₂O, brine, dried (MgSO₄), filtered, and concentrated. The crude material is purified by silica gel chromatography (gradient elution; R_{f} in 90:10 CH₂Cl₂:MeOH=0.51) to give a pale yellow oil. This is lyophilized to give the title compound as a solid (102 mg, 78%).

### 4-Chloro-N-[1-(4-ethyl-5-methoxy-1H-pyrazol-3-yl)ethyl]benzenesulfonamide

### Example 8

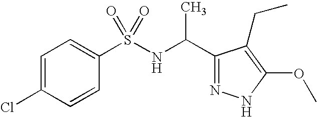

The procedure to generate Example 7 from Intermediate 4 may be applied to Intermediate 5 to yield Example 8.

### 4-chloro-N-[(1R)-1-(4-iodo-5-methylisoxazol-3-yl)-2-phenylethyl]benzene sulfonamide

### Example 9

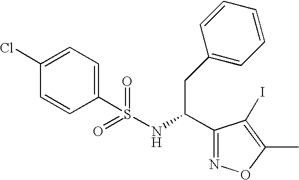

To a solution of N-[(1R)-1-benzyl-2-(methoxyimino)pent-3-yn-1-yl]-4-chlorobenzenesulfonamide (Intermediate 6a) in CH₃CN (20 mL) is treated with iodine (6210 mg). The resulting solution is stirred for 3 h in dark. The reaction mixture is poured into a saturated solution of sodium thiosulfate and is extracted with EtOAc (3×50 mL). The combined organic layers are dried over Na₂SO₄ and concentrated to yield crude product, which is purified using silica gel to afford 4-chloro-N-[(1R)-1-(4-iodo-5-methylisoxazol-3-yl)-2-phenylethyl]benzenesulfonamide (0.98 g, 45% over 2 steps).

### 4-chloro-N-[(1R)-1-(4-ethynyl-5-methylisoxazol-3-yl)-2-phenylethyl]benzene sulfonamide

### Example 10

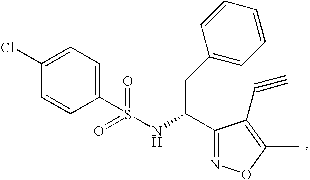

Example 10 may be prepared in two steps by using the compound obtained from Example 9 as described below:

### Step 1:

To a solution of 4-chloro-N-[(1R)-1-(4-iodo-5-methylisoxazol-3-yl)-2-phenylethyl]benzene-sulfonamide (Example 9, 100 mg) in DMF (0.7 mL) is treated with copper (I) iodide (7.6 mg), Et₃N (0.277 mL), ethynyl(trimethyl)silane (0.165 mL) and Pd(PPh₃)₄. The resulting solution is stirred for 45 min at 65° C. The reaction mixture is poured into a saturated solution of ammonia chloride and is extracted with EtOAc (3×50 mL). The combined organic layers are dried over Na₂SO₄ and concentrated to yield crude product, which is purified using silica gel to afford 4-chloro-n-((1R)-1-{5-methyl-4-[(trimethylsilyl)ethynyl]isoxazol-3-yl}-2-phenylethyl)benzene-sulfonamide (100 mg). M/Z 472.

### Step 2:

To a solution of 4-chloro-N-((1R)-1-{5-methyl-4-[(trimethylsilyl)ethynyl]isoxazol-3-yl}-2-phenylethyl)benzenesulfonamide (generated from step 1, 100 mg) in THF is added TBAF (0.317 mL). The resulting solution is stirred for 45 min. The reaction mixture is poured into a saturated solution of ammonia chloride and is extracted with EtOAc (3×50 mL). The combined organic layers are dried over Na₂SO₄ and concentrated to yield crude product, which is purified using silica gel to afford 4-chloro-N-[(1R)-1-(4-ethynyl-5-methylisoxazol-3-yl)-2-phenylethyl]benzenesulfonamide (Example 10, 24 mg, 28%).

### 4-chloro-N-[(1R)-1-(4-ethyl-5-methylisoxazol-3-yl)-2-phenylethyl]benzene sulfonamide

### Example 11

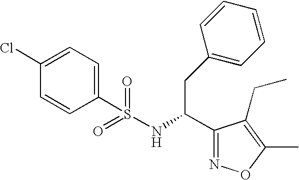

To a solution of 4-chloro-N-[(1R)-1-(4-ethynyl-5-methylisoxazol-3-yl)-2-phenylethyl]benzenesulfonamide (obtained from Example 10, 15 mg) in EtOH is added Pd/C (5 mg). The resulting solution is placed under the H₂ atmosphere for 45 min. The reaction mixture is filtered. The filtrate is dried and concentrated to yield crude product, which is purified using reverse phase HPLC to afford 4-chloro-N-[(1R)-1-(4-ethyl-5-methylisoxazol-3-yl)-2-phenylethyl]benzenesulfonamide (4 mg, 27%).

Example 12 and 13 may be prepared by using appropriate N-halo succinimide as represented below for Example 12.

### 4-chloro-N-[(1R)-1-(4-bromo-5-methylisoxazol-3-yl)-2-phenylethyl]benzene sulfonamide

### Example 12

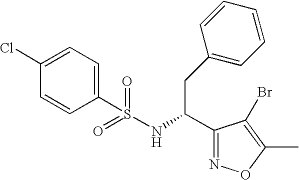

To a solution of 4-chloro-N-[(1R)-1-(5-methylisoxazol-3-yl)-2-phenylethyl]benzenesulfonamide (Intermediate 7, 17 mg) in DMF (0.20 mL) is treated with N-Bromosuccinimide (24 mg). The resulting solution is stirred for 3 h in dark at 110° C. The reaction mixture is poured into a saturated solution of sodium thiosulfate and is extracted with EtOAc (3×5 mL). The combined organic layers are dried over Na₂SO₄ and concentrated to yield crude product, which is purified using silica gel to afford 4-chloro-N-[(1R)-1-(4-bromo-5-methylisoxazol-3-yl)-2-phenylethyl]benzene sulfonamide (20 mg).

### N-[(1R)-1-(4-bromo-5-methoxyisoxazol-3-yl)-2-phenylethyl]-4-chlorobenzene sulfonamide

### Example 14

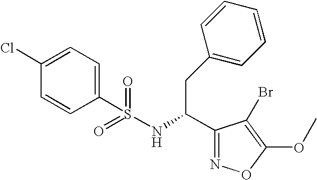

To a solution of 4-chloro-N-[(1R)-1-(5-methoxyisoxazol-3-yl)-2-phenylethyl]benzene-sulfonamide (Intermediate 8a, 100 mg) in DMF (1.3 mL) is treated with N-bromosuccinimide (224 mg). The resulting solution is stirred for 30 min in dark. The reaction mixture is poured into a saturated solution of sodium thiosulfate and is extracted with EtOAc (3×5 mL). The combined organic layers are dried over Na₂SO₄ and concentrated to yield crude product, which is purified using silica gel to afford N-[(1R)-1-(4-bromo-5-methoxyisoxazol-3-yl)-2-phenylethyl]-4-chlorobenzene sulfonamide (78 mg).

### 4-chloro-N-[(1R)-1-(4-ethyl-5-methoxyisoxazol-3-yl)-2-phenylethyl]benzene sulfonamide

### Example 15

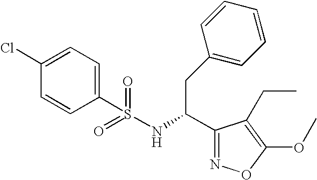

To a solution of 4-chloro-N-[(1R)-1-(4-ethyl-5-oxo-4,5-dihydroisoxazol-3-yl)-2-phenylethyl]benzenesulfonamide (Intermediate 8b) in diethyl ether (1.3 mL) and THF (1.3 mL) is treated with [(trimethylsilyl)methyl]diazane (0.15 mL, 1 M in diethyl ether). The resulting solution is stirred for 6 h. The reaction mixture is poured into water and was extracted with DCM (3×5 mL). The combined organic layers are dried over Na₂SO₄ and concentrated to yield crude product as yellow solid, which is purified on reverse phase HPLC to afford 4-chloro-N-[(1R)-1-(4-ethyl-5-methoxyisoxazol-3-yl)-2-phenylethyl]benzenesulfonamide (4.0 mg).

### 4-chloro-N-[1-(4-bromo-5-methylisoxazol-3-yl)ethyl]benzenesulfonamide

### Example 16

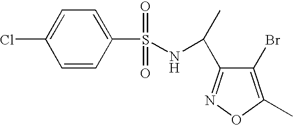

The procedure for Example 12 may be applied to 4-chloro-N-(1-(5-methylisoxazol-3-yl)ethyl)benzenesulfonamide (which may be prepared by applying procedure from step 1 of Example 17 to Intermediate 9) to yield the compound of Example 16.

### 4-chloro-N-[1-(4-iodo-5-methylisoxazol-3-yl)ethyl]benzenesulfonamide

### Example 17

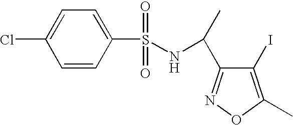

The title compound was generated in two steps from Intermediate 11 as described below:
Step 1: Cyclization: Isoxazole formation

### 4-chloro-N-[1-(5-methylisoxazol-3-yl)ethyl]benzenesulfonamide

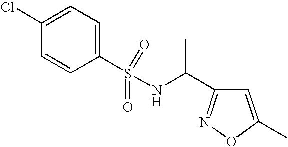

4-chloro-N-(1-methyl-2-oxopent-3-yn-1-yl)benzenesulfonamide (Intermediate 9, 285 mg, 1 mmol), hydroxylamine hydrochloride (76 mg, 1.1 mmols) and ammonium acetate (90 mg, 1.1 mmol) were taken in a microwave tube equipped with a stir bar and ethanol (2 mL) and water (1 mL) are added to it. The resultant mixture was heated in the microwave at 150° C. for 2 hours. The contents were concentrated and the solution was reconstituted in ethyl acetate and washed with water and then brine. The resultant mixture was dried over Na₂SO₄ (anhy.), filtered, evaporated and the crude solid was purified by column chromatography using a gradient of 5% to 100% ethyl acetate in hexanes to isolate the desired product as an off white solid (0.216 mg, 72%). M/Z 300.

### Step 2: Iodination

4-chloro-N-[1-(5-methylisoxazol-3-yl)ethyl]benzenesulfonamide (0.133 mg, 0.44 mmols) and iodine crystals (0.113 mg, 0.44 mmols) were taken in a round bottom flask equipped with a stir bar and a water condenser. Concentrated nitric acid (0.5 mL) was added to it and the resultant mixture was heated at 80° C. for 45 minutes. The reaction mixture was cooled to room temperature and poured over ice and partitioned between ethyl acetate and ice-water. Solid sodium bisulfite was added to the biphasic solution to destroy any unreacted iodine. The organic layer was separated and washed with brine and dried over Na₂SO₄ (anhy.), filtered and concentrated to generate a yellowish solid which was spurifed by column chromatography using a gradient of 5% to 50% ethyl acetate in hexanes to obtain pure desired product, Example 17 (0.122 mg, 66%)

### 4-Chloro-N-[1-(5-ethyl-4-iodo-isoxazol-3-yl)-ethyl]-benzenesulfonamide

### Example 18

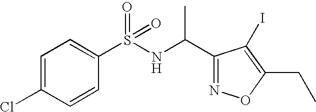

Application of the procedure for Example 17 was applied to 4-chloro-N-[1-(5-ethyl-isoxazol-3-yl)-ethyl]benzenesulfonamide (Intermediate 10c) to yield compound of Example 18.

### 4-Chloro-N-[1-(5-ethyl-4-ethynyl-isoxazol-3-yl)-ethyl]-benzenesulfonamide

### Example 19

Example 19 was generated from Example 18 in two steps as described below

### Step 1:

### 4-Chloro-N-[1-(5-ethyl-4-trimethylsilanylethynyl-isoxazol-3-yl)-ethyl]-benzenesulfonamide

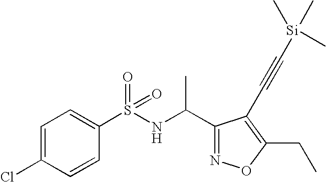

Under a nitrogen purge, 4-chloro-N-[1-(5-ethyl-4-iodo-isoxazol-3-yl)-ethyl]-benzenesulfonamide (Example 18, 0.33 g; 0.00075 mol), trimethylsilylacetylene (0.15 g; 0.0015 mol), tetrakis(triphenylphosphine) palladium (0) (0.04 g; 5 mol %), and cupric iodide (0.014 g; 10 mol %) were added to a solvent mixture of dimethylformamide (6 mL) and triethylamine (2 mL) in a 50 μL 3-neck round-bottomed flask. The reaction mixture was heated to 70 C and maintained for 1 h. The reaction mixture was filtered through Celite, and the filter cake was washed with DMF. Using high vacuum, the solvent was removed. The crude residue was purified by column chromatography using a gradient of 0%-35% ethyl acetate in hexanes to obtain the desired product (0.21 g). ¹H NMR (300 MHz, chloroform-d) δ ppm 0.29 (s, 9H), 1.16-1.21 (t, 3H), 1.54-1.57 (d, 2H), 1.61 (s, 3H), 2.44-2.54 (q, 2H), 4.77-4.84 (m, 1H), 5.27-5.33 (d, 1H), 7.30-7.35 (dd, 2H), 7.63-7.68 (dd, 2H). M/Z=411.

### Step 2:

4-Chloro-N-[1-(5-ethyl-4-trimethylsilanylethynyl-isoxazol-3-yl)-ethyl]-benzenesulfonamide from step 1 (0.21 g; 0.0005 mol) was dissolved in ca 5 mL THF in 50 mL 3-neck round-bottomed flask under a nitrogen purge. Tetrabutylammonium fluoride (1.3 mL; 1 N in THF; 10 equiv) was added dropwise. The reaction mixture was then stirred 2 h at room temperature. The solvent was removed under reduced pressure and the residue partitioned between ethyl acetate and water. The organic layer was washed with water twice, and then with saturated sodium chloride solution. Upon drying with magnesium sulfate, removal of solvent under reduced pressure provided the desired product. The product was purified by column chromatography using a gradient of 0% to 35% ethyl acetate in hexanes to obtain the desired product (21 mg).

### 4-Chloro-N-[1-(4,5-diethyl-isoxazol-3-yl)-ethyl]-benzenesulfonamide

### Example 20

4-Chloro-N-[1-(5-ethyl-4-ethynyl-isoxazol-3-yl)-ethyl]-benzenesulfonamide (Example 19, 0.011 g; 0.0003 mol) and 10% palladium-on-carbon (0.0017 mol; 5 mol %) were added to ethanol (10 mL). A hydrogen filled balloon was placed over an inlet, and the contents of the flask are alternatively put under vacuum and then under a hydrogen atmosphere. After three such cycles, the reaction was kept under a hydrogen atmosphere. After reacting for 16 h, the reduction is only partially complete, leading to a mixture of the desired diethyl compound, along with the ethyl, vinyl analog. Another 5 mol % of Pd/C is charged to the system and the reaction was allowed to continue under hydrogen atmosphere. The resulting crude product was separated by RP-HPLC to obtain the desired compound (2 mg).

The sequence of reactions for Examples 18-20 were applied to intermediate 11e and 12e to yield compounds of Examples 21-25.
The intermediates listed in Table 2 were prepared as described below:

TABLE 2

| | | | | Prepared |
|---|---|---|---|---|
| | | | | from |
| Int # | Structure | 1H NMR | M/Z | S.M # |
| 1 | 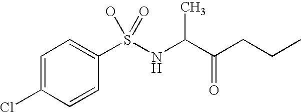 | (400 MHz, CDCl3-D3) δppm 0.79 (t, J = 7.45 Hz, 3H)1.34 (d, J = 7.33 Hz, 3H)1.42-1.53 (m, 2H)2.23 (m, 1H) 2.42 (m, 1H)3.88-3.96 (m, 1H)5.61 (m, 1H) 7.45 (m, 2H)7.76 (m, 2H) | 289 | 1 |
| 2 | 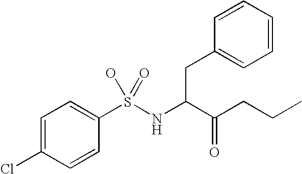 | (400 MHz, DMSO-D6) δppm 0.75 (t, J = 7.33 Hz, 3H)1.29-1.39 (m, 2H)2.44 (m, 2H) 2.50-2.56(m, 1H) 2.90 (m, 1H)3.96 (m, 1H) 7.06-7.16(m, 5H) 7.42-7.51 (m,4H) 8.47 (m, 1H) | 365 | 2 |
| 3 | 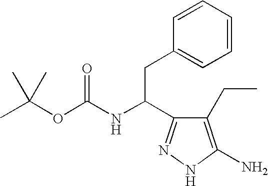 | (400 MHz, DMSO-D6) δppm 0.82 (t, J = 7.07 Hz, 3H)1.29 (s, 9H) 2.10-2.22 (m, 2H) 2.93 (m, 2H)4.27 (broad s, 1H)4.63-4.75 (m, 1H) 6.92(broad s, 1H) 7.10-7.18(m, 3H) 7.18-7.29 (m,2H) 10.90 (broad s, 1H) | 330 | 3 |
| 4 | 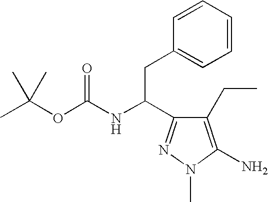 | — | 344 | 3 |
| 5 | 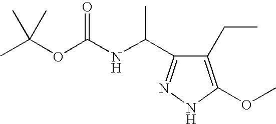 | — | 269 | 4 |
| 6 | 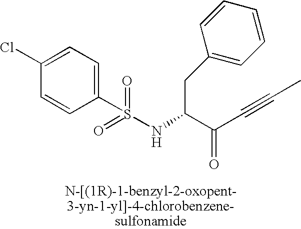 | — | 361 | 2′ (R-isomer) |
| 7 | 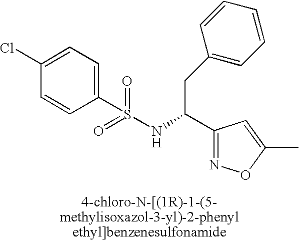 | (300 MHz, CDCl₃)δ 7.52 (2H, m), 7.28 (2H,d), 7.18 (3H, m), 6.98(2H, m), 5.74 (1H, s),5.22 (1H, d), 4.64 (1H,m), 3.14 (1H, dd), 2.98(1H, dd), 2.32 (3H, s) | 376 | 2 |
| 8 | 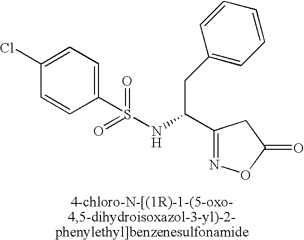 | | 378 | 5 |
| 9 | 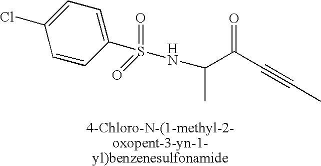 | — | 285 | 1 |
| 10 | 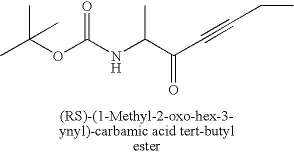 | (300 MHz, DMSO-d₆) δ 7.38 (d, J = 7.1 Hz,1H), 3.99-3.92 (m, 1H),2.47-2.40 (m, 2H), 1.39(s, 9H), 1.21-1.11 (m,6H) | [(M − 100) + 1]/Z = 126 | 6 |
| 11 | 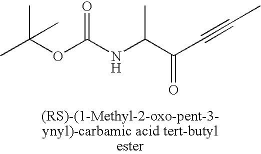 | (300 MHz, DMSO-d₆) δ 7.38 (d, J = 7.1 Hz,1H), 4.02-3.92 (m, 1H),2.07 (s, 3H), 1.39 (s, 9H),1.20 (d, J = 7.4 Hz, 3H) | [(M − 100) + 1]/Z = 112 | 6 |

### Preparation of Intermediates:

### 4-Chloro-N-(1-methyl-2-oxopentyl)benzenesulfonamide (Intermediate 1)

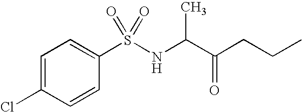

An oven-dried 250 mL round bottom flask was evacuated while hot and allowed to cool under N₂. The flask was charged with N²-[(4-chlorophenyl)sulfonyl]-N¹-methoxy-N¹-methylalaninamide (Starting material 1, 3.13 g, 10.20 mmol), evacuated and back-filled with N₂. Anhydrous THF (20 mL) was added, and the solution was cooled to 0° C. n-Propyl magnesium chloride (2.0 M in diethyl ether; 12.0 mL, 24.0 mmol) was added dropwise, and the solution slowly allowed to warm to room temperature. After stirring at room temperature overnight, the reaction was quenched with saturated aqueous NH₄Cl (5 mL). The mixture was partitioned between EtOAc and H₂O, and the aqueous layer further extracted with EtOAc. The combined organics were washed with H₂O, brine, dried (MgSO₄), filtered, and concentrated. The crude material was purified by silica gel chromatography (gradient elution; R_{f} in 70:30 hexanes:EtOAc=0.36) to give a pale yellow solid (1.99 g, 67%). ¹H NMR (400 MHz, CDCl₃) δ 0.79 (t, J=7.45 Hz, 3H) 1.34 (d, J=7.33 Hz, 3H) 1.42-1.53 (m, 2H) 2.23 (m, 1H) 2.42 (m, 1H) 3.88-3.96 (m, 1H) 5.61 (m, 1H) 7.45 (m, 2H) 7.76 (m, 2H). M/Z-289.

### N-(1-Benzyl-2-oxopentyl)-4-chlorobenzenesulfonamide: (Intermediate 2)

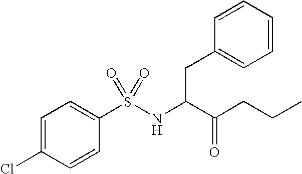

Application of the above procedure described for preparation of intermediate-1 was applied to N-[(4-chlorophenyl)sulfonyl]-N-methoxy-N-methylphenylalaninamide (Starting Material 2) to yield the desired Intermediate 2 in 84% yield as a pale yellow solid. M/Z 365.

### N-(1-Benzyl-3-ethyl-2,4-dioxohexyl)-4-chlorobenzenesulfonamide (Intermediate 2a)

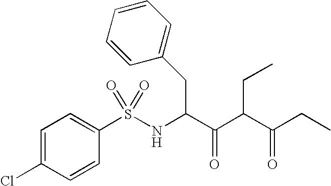

An oven-dried 100 mL round bottom flask was evacuated while hot and allowed to cool under N₂. The flask was twice further evacuated and backfilled with N₂, and charged with anhydrous diisopropylamine (0.90 mL, 6.4 mmol) and anhydrous THF (10 mL). This solution was cooled to 0° C., and n-BuLi (2.5 M solution in hexanes; 2.50 mL, 6.30 mmol) was added dropwise. The resulting solution was allowed to stir at 0° C. for 30 min, and then cooled to −78° C. A solution of N-(1-benzyl-2-oxopentyl)-4-chlorobenzenesulfonamide (Intermediate 2, 739 mg, 2.02 mmol) in anhydrous THF (3.0 mL) was added dropwise, and the resulting solution allowed to stir at −78° C. for 45 min. Propionyl chloride (0.20 mL, 2.3 mmol) was then added, and then after 30 min more at −78° C. the mixture was quenched with HOAc (0.4 mL) and allowed to warm to room temperature. The mixture was partitioned between EtOAc and H₂O, and the aqueous layer was further extracted with EtOAc. The combined organics were washed with H₂O, brine, dried (MgSO₄), filtered, and concentrated. The crude material was purified by silica gel chromatography (gradient elution; R_{f} in 80:20 hexanes:EtOAc=0.27) to give a colorless oil (761 mg, 89%), which appears to exist as a mixture of keto/enol tautomers as well as enol E/Z isomers by NMR. M/Z=421.

### tert-Butyl[1-(5-amino-4-ethyl-1H-pyrazol-3-yl)-2-phenylethyl]carbamate: (Intermediate 3)

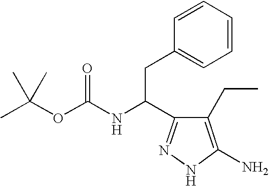

A 50 mL round bottom flask was charged with tert-butyl (1-benzyl-3-cyano-2-oxopentyl)carbamate (Starting material 3, 1.07 g, 3.38 mmol) and EtOH (15 mL). Hydrazine monohydrate (330 μL, 6.80 mmol) was added, and the mixture was heated to reflux overnight. On cooling, the volatile components were removed under reduced pressure, and the residue purified by silica gel chromatography (gradient elution; R_{f} in 90:10 CH₂Cl₂:MeOH=0.29) to give a colorless foam (641 mg, 57%). M/Z 330.

### N-(1-{5-Amino-1-[(4-chlorophenyl)sulfonyl]-4-ethyl-1H-pyrazol-3-yl}-2-phenylethyl)-4-chlorobenzenesulfonamide (Intermediate 3a)

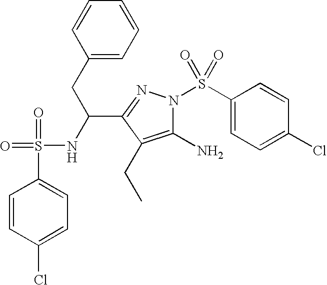

A 50 mL roundbottom flask was charged with tert-butyl[1-(5-amino-4-ethyl-1H-pyrazol-3-yl)-2-phenylethyl]carbamate (intermediate 3, 1.50 mmol) and 4 N HCl/dioxane (6 mL). The mixture was allowed to stir at room temperature overnight. The volatile components were removed under reduced pressure, and the reside was dissolved in CH₂Cl₂ (10 mL) and NEt₃ (2.00 mL, 14.3 mmol). 4-Chlorobenzenesulfonyl chloride (1.03 g, 4.74 mmol) was added, and the mixture was allowed to stir at room temperature for 6 h. The mixture was partitioned between CH₂Cl₂ and H₂O, and the aqueous layer was further extracted with CH₂Cl₂. The combined organics were washed with H₂O, brine, dried (MgSO₄), filtered, and concentrated. The crude material was purified by silica gel chromatography (gradient elution; R_{f} in 70:30 hexanes:EtOAc=0.33) to give an oil (502 mg, 58%). M/Z=579. 1H NMR (400 MHz, DMSO-D6) δ ppm 0.49 (t, J=7.45 Hz, 3H) 1.77-1.88 (m, 2H) 2.69 (m, 1H) 2.80-2.90 (m, 1H) 4.12-4.22 (m, 1H) 5.83 (broad s, 2H) 6.63 (m, 2H) 6.95 (m, 2H) 7.04 (m, 1H) 7.41-7.47 (m, 2H) 7.59 (m, 2H) 7.75 (m, 2H) 7.83-7.91 (m, 2H) 8.48 (m, 1H).

### tert-Butyl[1-(5-amino-4-ethyl-1-methyl-1H-pyrazol-3-yl)-2-phenylethyl]carbamate: (Intermediate 4)

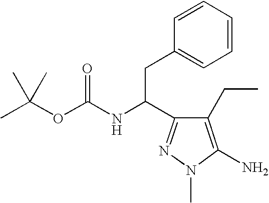

A 50 mL round bottom flask was charged with tert-butyl (1-benzyl-3-cyano-2-oxopentyl)carbamate (Intermediate 3, 629 mg, 1.99 mmol) and methylhydrazine (4.00 mL, 75.2 mmol). The resulting mixture was heated at 80° C. overnight. On cooling, the excess methylhydrazine was removed under reduced pressure to give a yellow oil. This crude material was used without further purification. M/Z 344.

### tert-Butyl[1-(4-ethyl-5-methoxy-1H-pyrazol-3-yl)ethyl]carbamate (Intermediate 5)

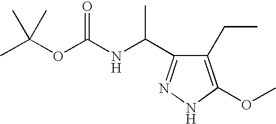

A test tube equipped with a stir bar was charged with tert-butyl[1-(4-ethyl-5-oxo-2,5-dihydro-1H-pyrazol-3-yl)ethyl]carbamate (Starting material 4, 261 mg, 1.02 mmol) and Cs₂CO₃ (507 mg, 1.56 mmol). Anhydrous DMF (1.5 mL) was added, and the resulting mixture allowed to stir at room temperature for 10 min. MeI (75 μL, 1.2 mmol) was then added, followed by additional DMF (0.5 mL). The mixture was allowed to stir at room temperature overnight. The mixture was partitioned between EtOAc and H₂O, and the aqueous layer was further extracted with EtOAc. The combined organics were washed with brine, dried (MgSO₄), filtered, and concentrated under reduced pressure. The crude material (a mixture of materials) was used directly without further purification. M/Z 269.

### N-[(1R)-1-Benzyl-2-oxopent-3-yn-1-yl]-4-chlorobenzenesulfonamide (Intermediate 6)

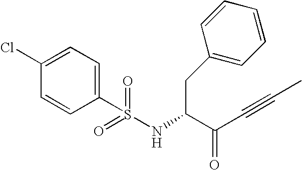

To a solution of N-[(4-chlorophenyl)sulfonyl]-N-methoxy-N-methyl-D-phenylalaninamide (Starting Material 2, 2 g, 5.22 mmol) in THF (20 mL) was added propynyl magnesium bromide (21 mL, 0.5 M in THF). The resulting solution was stirred overnight. The reaction mixture was poured into water and extracted with EtOAc (3×50 mL). The combined organic layers were dried over Na₂SO₄ and concentrated to yield crude product, which was purified on silica gel to afford N-[(1R)-1-benzyl-2-oxopent-3-yn-1-yl]-4-chlorobenzenesulfonamide (1.6 g, 85%). M/Z 361.

### N-[(1R)-1-Benzyl-2-(methoxyimino)pent-3-yn-1-yl]-4-chlorobenzenesulfonamide (intermediate 6a)

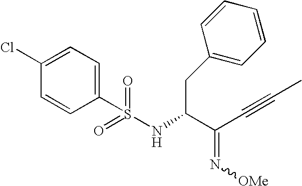

To a solution of N-[(1R)-1-benzyl-2-oxopent-3-yn-1-yl]-4-chlorobenzenesulfonamide (Intermediate 6, 1.6 g, 4.4 mmol) in MeOH (12 mL) was added pyridine (1.3 mL), sodium sulfate (1.88 g) and O-methylhydroxylamine(aminooxy)methane hydrochloride salt (733 mg). The resulting solution was stirred for 3 h. The reaction mixture was poured into water and extracted with EtOAc (3×50 mL). The combined organic layers were dried over Na₂SO₄ and concentrated to yield crude product, which was used directly in the next step (1.6 g, 85%). M/Z 390.

### 4-Chloro-N-[(1R)-1-(5-methylisoxazol-3-yl)-2-phenylethyl]benzenesulfonamide (Intermediate 7)

Intermediate 7 was prepared from Example 9 (which was prepared from Starting Material 2)

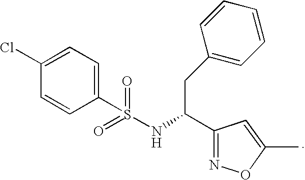

To a solution of 4-chloro-N-[(1R)-1-(4-iodo-5-methylisoxazol-3-yl)-2-phenylethyl]-benzenesulfonamide (Example 9, 100 mg) in THF (2 mL) was added n-BuLi (0.348 mL) at −78° C. The resulting solution was stirred for 45 min. The reaction mixture was poured into a saturated solution of ammonia chloride and extracted with EtOAc (3×50 mL). The combined organic layers were dried over Na₂SO₄ and concentrated to yield crude product, which was purified using silica gel to afford 4-chloro-N-[(1R)-1-(5-methylisoxazol-3-yl)-2-phenylethyl]benzene-sulfonamide (0.015 g, 20%).

### 4-Chloro-N-[(1R)-1-(5-oxo-4,5-dihydroisoxazol-3-yl)-2-phenylethyl]benzene sulfonamide (Intermediate 8)

Intermediate 8 was generated in two steps from commercially available D-alanine:

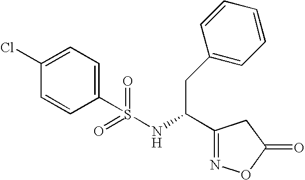

To solution of (4R)-4-{[(4-chlorophenyl)sulfonyl]amino}-3-oxo-5-phenylpentanoate (Starting Material 5 which was generated from Starting Material 2a′, 1.42 g) in EtOH (33 mL) was added hydroxylamine hydrochloride salt (723 mg) and sodium acetate (1.13 g). The suspension was heated at reflux for 3 h. The reaction mixture was poured into water and extracted with EtOAc (3×20 mL). The combined organic layers were dried over Na₂SO₄ and concentrated to yield crude product as yellow solid, which was purified on silica gel to afford 4-chloro-N-[(1R)-1-(5-oxo-4,5-dihydroisoxazol-3-yl)-2-phenylethyl]benzenesulfonamide (1.2 g, 90%). M/z 378.

### 4-Chloro-N-[(1R)-1-(5-methoxyisoxazol-3-yl)-2-phenylethyl]benzenesulfonamide (Intermediate 8a)

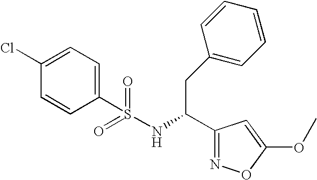

A solution of 4-chloro-N-[(1R)-1-(5-oxo-4,5-dihydroisoxazol-3-yl)-2-phenylethyl]benzene-sulfonamide (Intermediate 8, 100 mg) in diethyl ether (1.3 mL) was treated with [(trimethylsilyl)methyl]diazane (0.15 mL, 1 M in diethyl ether). The resulting solution was stirred for 6 h. The reaction mixture was poured into water and extracted with DCM (3×5 mL). The combined organic layers were dried over Na₂SO₄ and concentrated to yield crude product as yellow solid, which was purified on reverse phase HPLC to afford 4-chloro-N-[(1R)-1-(5-methoxyisoxazol-3-yl)-2-phenylethyl]benzenesulfonamide (22 mg, 22%). M/z 392. ¹H NMR (300 MHz, CDCl₃) δ 7.48 (2H, δ), 7.32 (2H, δ), 7.14 (3H, m), 6.94 (2H, m), 4.94 (2H, m), 4.49 (1H, m), 3.84 (3H, s), 3.08 (1H, dd), 2.91 (1H, dd).

### 4-Chloro-N-[(1R)-1-(4-ethyl-5-oxo-4,5-dihydroisoxazol-3-yl)-2-phenylethyl]benzene sulfonamide (Intermediate 8b)

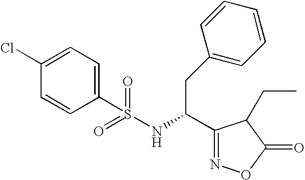

To a solution of 4-chloro-N-[(1R)-1-(5-oxo-4,5-dihydroisoxazol-3-yl)-2-phenylethyl]benzenesulfonamide (Intermediate 8, 100 mg) in EtOH (5.5 mL) was added acetaldehyde (0.7 mL). The reaction mixture was stirred for 4 h to yield 4-chloro-N-{(1R)-1-[(4Z)-4-ethylidene-5-oxo-4,5-dihydroisoxazol-3-yl]-2-phenylethyl}benzenesulfonamide. The crude mixture was concentrated and redissolved in EtOH, which was treated with sodium boron hydride (excess, ˜500 mg). After bubbling ceased, the reaction mixture was diluted with a solution of hydrochloride acid and extracted with DCM. The combined organic layers were dried over Na₂SO₄ and concentrated to yield crude product as yellow solid, which was used directly in the next step. M/Z 406.

### 4-Chloro-N-(1-methyl-2-oxopent-3-yn-1-yl)benzenesulfonamide (Intermediate 9)

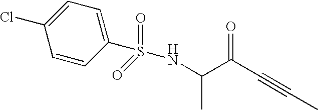

Intermediate 9 was generated in 2 steps from commercially available alanine as described for preparation of Intermediate 6. M/Z 285.
Intermediates 10 and 11 were prepared by the procedure outlined below for intermediate 11:

### (DL)-(1-Methyl-2-oxo-pent-3-ynyl)-carbamic acid tert-butyl ester (Intermediate 11)

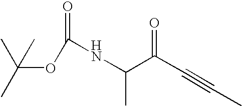

The Grignard reagent, prop-1-ynyl magnesium bromide (155 mL, 77.6 mmol) was added to a solution of the N²-(tert-butoxycarbonyl)-N¹-methoxy-N¹-methylalaninamide (Starting Material 6, 9.0 g, 38.8 mmol) at 0° C. and the resulting mixture stirred at RT overnight. The reaction mixture was poured into water and extracted with EtOAc. The combined organic layer was washed with brine and dried. Evaporation of the solvent gave a crude material that was purified by flash column chromatography on silica gel using hexanes/EtOAc (8:2) followed by recrystallization from Et₂O/n-pentane to give the title compound as a cream colored solid (5.16 g, 63% yield). ¹H NMR (300 MHz, DMSO-d₆) δ 7.38 (d, J=7.1 Hz, 1H), 4.02-3.92 (m, 1H), 2.07 (s, 3H), 1.39 (s, 9H), 1.20 (d, J=7.4 Hz, 3H). [(M−100)+1]/Z=112.

### (DL)-(1-Methyl-2-oxo-hex-3-ynyl)-carbamic acid tert-butyl ester (Intermediate 10)

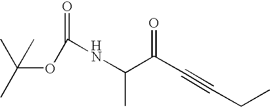

Application of the procedure to generate Intermediate 11 to Butynyl magnesium bromide [which was prepared from EtMgBr (34.5 mL, 103.4 mmol, 3.0 M in Et₂O) and 1-butyne (saturated solution in Et₂O)] and N²-(tert-butoxycarbonyl)-N¹-methoxy-N¹-methylalamamide (Starting Material 6) yielded Intermediate 12 (6.6 g, 57% yield).

### [1-(5-Ethyl-isoxazol-3-yl)-ethyl]-carbamic acid tert-butyl ester (Intermediate 10a)

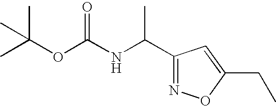

The procedure for converting Intermediate 6 to Intermediate 6a was used to convert Intermediate 10 to Intermediate 10a. ¹H NMR (300 MHz, Chloroform-D) δ ppm 1.22-1.28 (t, 3H), 1.45 s, 9H), 1.48-1.53 (d, 3H), 2.26 (s, 1H) 2.62-2.71 (q, 2H), 4.98 (br s), 5.98 (s, 1H). M/Z 281 (M+CH3CN).

### 1-(5-Ethyl-isoxazol-3-yl)-ethylamine hydrochloride (Intermediate 10b)

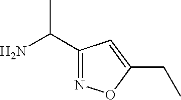

Under a nitrogen purge, 1-(5-ethyl-isoxazol-3-yl)-ethyl]-carbamic acid tert-butyl ester (0.12 g; 0.001 mol) was dissolved in ca 5 mL dioxane. In a single portion, 1 mL 4N HCl/dioxane (0.004 mol) was added and the reaction mixture stirred at room temp for about 4 h. The solvent was removed under reduced pressure, and the resulting hydrochloride salt was used in the subsequent step. M/Z=141.

### 4-Chloro-N-[1-(5-ethyl-isoxazol-3-yl)-ethyl]-benzenesulfonamide (Intermediate 10c)

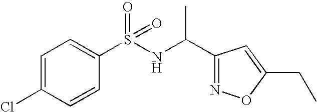

Under a nitrogen purge, 1-(5-ethyl-isoxazol-3-yl)-ethylamine hydrochloride (Intermediate 10b, 0.28 g; 0.002 mol) was dissolved in THF (15 mL) in a 50 mL round-bottomed flask. DIEA (0.38 mL; 0.0022 mol) was added in a single portion, and the flask was cooled in an ice-acetone bath. 4-Chlorosulfonyl chloride (0.38 g; 0.002 mol), dissolved in THF (5 mL), was added dropwise. After allowing the reaction mixture to warm to ambient temperature, stirring was continued for another 16 h. Solvent was removed under reduced pressure, and the resulting residue was partitioned between ethyl acetate and water. The organic layer was washed with water, and then with saturated sodium chloride solution. After drying over magnesium sulfate, solvent was removed under reduced pressure. The resulting crude material was purified by column chromatography using a gradient of 15% to 50% ethyl acetate in hexanes to afford 0.31 g of the desired product. M/Z=315.

Intermediates 11d and 12d may be prepared from 11 and 12 respectively, by the method described below for intermediate 11.

### tert-Butyl[1-(5-methylisothiazol-3-yl)ethyl]carbamate (Intermediate 11d)

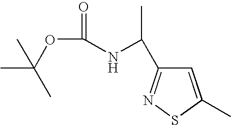

Hydroxylamine-O-sulfonic acid (125 mg, 1.1 mmol) was dissolved in methanol (1 mL) and tert-butyl (1-methyl-2-oxopent-3-yn-1-yl)carbamate (Intermediate 11, 210 mg, 1 mmol) in methanol (1 mL) was added to it. The resultant mixture was stirred until Intermediate 11 was determined to have been consumed based on LC-MS. Sodium bicarbonate (92.4 mg, 1.1 mmols) was added in small portions followed by sodium hydrosulfide (0.73 mL, 1.5 M), which was added slowly and the resultant mixture was allowed to stir at room temperature overnight. The reaction mixture was concentrated and partitioned between ethyl acetate and water and the organic layer dried (Na₂SO₄), filtered, concentrated and subjected to flash chromatography using a gradient of 10% ethyl acetate in hexanes to 100% ethyl acetate to obtain the desired product (57 mg, 24%). M/Z+Na 265.

### tert-Butyl[1-(5-ethylisothiazol-3-yl)ethyl]carbamate (Intermediate 10d)

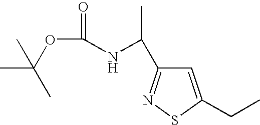

The procedure for the preparation of Intermediate 11d from Intermediate 11 was applied to Intermediate 10 to generate Intermediate 10d. M/Z+Na 279.

### 4-Chloro-N-[1-(5-methylisothiazol-3-yl)ethyl]benzenesulfonamide (Intermediate 11e)

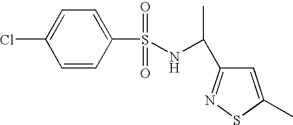

tert-butyl[1-(5-methylisothiazol-3-yl)ethyl]carbamate (Intermediate 11d, 57 mg, 0.236 mmols) was dissolved in dioxane (0.3 mL) and 4M HCl/dioxane (0.6 mL) was added to it. The resultant mixture was stirred at room temperature. As the reaction progressed, a white precipitate formed. When the starting material was gone, the reaction mixture was concentrated and dried in vacuo overnight. To the dried solid, DCM (1 mL) was added and the mixture was cooled to 0° C. and TEA (0.072 mL, 0.526 mmols) added followed by p-chloro phenyl sulfonyl chloride (55 mg, 0.235 mmols) dissolved in DCM (1 mL). The resultant mixture was stirred at 0° C. for 50 minutes. The reaction mixture was concentrated in vacuo and the resultant mixture partitioned between ethyl acetate and water. The organic layer was dried (anhydrous Na₂SO₄), filtered and concentrated on a rotary evaporator. The product thus obtained was purified by flash chromatography using a gradient of 10% ethyl acetate in hexanes to 100% ethyl acetate to isolate the desired product as an off white powder (49.5 mg, 66.6%) ¹HNMR (CDCl3); δ 7.75 (d, 2H), 7.40 (d, 2H), 6.59 (s, 1H), 5.66 (d, 1H), 4.56 (m, 1H), 2.50 (s, 3H), 1.48 (d, 3H). M/Z 316.83, M+Na 339.

### 4-Chloro-N-[1-(5-ethylisothiazol-3-yl)ethyl]benzenesulfonamide (Intermediate 12e)

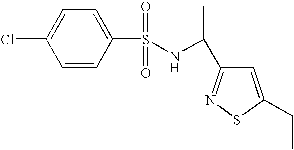

The method to convert Intermediate 11d into Intermediate 11e was applied to Intermediate 10d to afford Intermediate 12e. ¹H NMR (300 MHz, CDCl₃) δ 1.20 (t, 3H) 1.42 (d, 3H) 2.74 (q, 2H) 4.61 (m, 1H) 6.41 (d, 1H) 6.62 (s, 1H) 7.29 (d, 2H) 7.66 (d, 2H). M/Z 331.

### Preparation of Starting Materials:

### N²-[(4-Chlorophenyl)sulfonyl]-N¹-methoxy-N¹-methylalaninamide (Starting material 1)

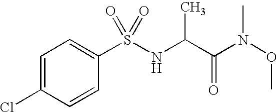

A 250 mL round bottom flask containing N-[(4-chlorophenyl)sulfonyl]alanyl chloride (Starting Material 1b, 32.9 mmol) was charged with N,O-dimethylhydroxylamine hydrochloride (3.94 g, 40.39 mmol) and CH₂Cl₂ (70 mL). The suspension was cooled to 0° C., and then triethylamine (12.0 mL, 86.1 mmol) was added dropwise over 10 min. After slowly warming to room temperature over the course of 4 h, the mixture was partitioned between CH₂Cl₂ and H₂O. The aqueous layer was further extracted with CH₂Cl₂, and the combined organics were washed with brine, dried (MgSO₄), filtered, and concentrated. The crude material was recrystallized from MeOH to give a crystalline solid (6.79 g, 67%). M/Z=306. ¹H NMR (400 MHz, CDCl₃) δ 1.31 (d, J=7.07 Hz, 3H) 2.99 (s, 3H) 3.58 (s, 3H) 4.35 (m, 1H) 5.55 (m, 1H) 7.45 (m, 2H) 7.77 (m, 2H).

### N-[(4-Chlorophenyl)sulfonyl]alanyl chloride (Starting material 1b)

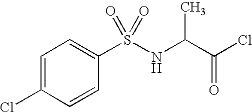

A 250 mL round bottom flask was charged with N-[(4-chlorophenyl)sulfonyl]alanine (8.69 g, 32.95 mmol) and SOCl₂ (30 mL). The mixture was heated at 80° C. overnight. On cooling, the excess SOCl₂ was removed under reduced pressure to give a solid material. This was used without further purification. ¹H NMR (400 MHz, CDCl₃) δ 1.52 (d, J=7.33 Hz, 3H) 4.34 (m, 1H) 5.21-5.31 (m, 1H) 7.50 (m, 2H) 7.79 (m, 2H).

### N-[(4-Chlorophenyl)sulfonyl]alanine: (Starting material 1a)

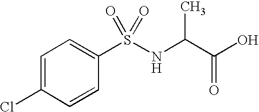

The titled starting material was prepared by the known literature reference procedure by DeRuiter, Jack et al, J. Pharm. Sci.; 76; 2; 1987; 149-152.

### N-[(4-Chlorophenyl)sulfonyl]-N-methoxy-N-methylphenylalaninamide (Starting material 2)

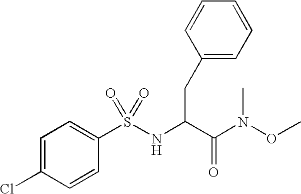

The titled Starting Material 2 was generated in a two step sequence from Starting Material 2a (63% yield over two steps) by methods analogous to those described for generation of Starting Material 1 from 1a. 1H NMR (400 MHz, DMSO-D6) δ ppm 2.61 (m, 1H) 2.83 (m, 1H) 2.91 (s, 3H) 3.55 (s, 3H) 4.38 (m, 1H) 7.07 (m, 1H) 7.09 (m, 1H) 7.14-7.22 (m, 3H) 7.44-7.53 (m, 4H) 8.48 (m, 1H). M/Z=382.

### N-[(4-Chlorophenyl)sulfonyl]phenylalanyl chloride (Starting material 2b)

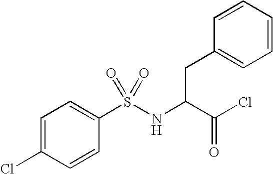

The titled starting material was generated from N-[(4-chlorophenyl)sulfonyl]-phenylalanine (Starting Material 2a) by method analogous to that for generation of Starting Material 1b from 1a to obtain an oily residue which was used without further purification.

### N-[(4-Chlorophenyl)sulfonyl]phenylalanine (starting material 2a)

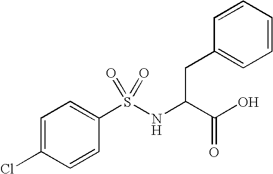

Starting material 2a and 2a′ (R isomer) was prepared by a method analogous to that for generating Starting Material 1a and was used without further purification. M/Z 339.

### tert-Butyl (1-benzyl-3-cyano-2-oxopentyl)carbamate (starting material 3)

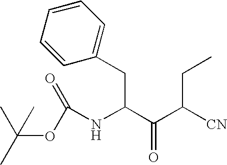

An oven-dried 250 mL round bottom flask was evacuated while hot and allowed to cool under N₂. The flask was charged with anhydrous THF (40 μL) and cooled to −78° C. A solution of n-BuLi (2.5 M in hexanes; 20.0 mL, 50.0 mmol) was added, followed by butyronitrile (4.40 mL, 50.6 mmol). After 1 h at −78° C., commercially available BOC-Phe-OMe (4.34 g, 15.5 mmol) was added in one portion. The reaction was allowed to warm to −50° C. After 90 min at this temperature, the reaction was quenched with glacial HOAc (3 mL) and allowed to warm to rt. The mixture was partitioned between EtOAc and H₂O, and the aqueous layer was further extracted with EtOAc. The combined organics were washed with H₂O, brine, dried (MgSO₄), filtered, and concentrated. The crude material was purified by silica gel chromatography (gradient elution; R_{f} in 80:20 hexanes:EtOAc=0.35) to give a pale yellow oil that solidifies on standing (3.90 g, 79%). M/Z=316. 1H NMR appeared to indicate a mixture of isomers is present—the material is carried directly to the next step.

### tert-Butyl[1-(4-ethyl-5-oxo-2,5-dihydro-1H-pyrazol-3-yl)ethyl]carbamate (starting material 4)

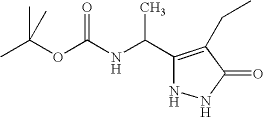

A 50 ml round bottom flask was charged with Isopropyl 4-[(tert-butoxycarbonyl)amino]-2-ethyl-3-oxopentanoate (Starting Material 4a) (1.91 g, 6.34 mmol) and MeOH (15 μL). The solution was treated with hydrazine monohydrate (1.25 mL, 25.8 mmol) and allowed to stir at room temperature overnight before the volatile components are evaporated under reduced pressure. The residue was redissolved in ˜10 mL MeOH and reconcentrated (to remove residual unreacted hydrazine), giving a colorless, viscous oil which was used without further purification. M/Z 255.

### Isopropyl 4-[(tert-butoxycarbonyl)amino]-2-ethyl-3-oxopentanoate (Starting Material 4a)

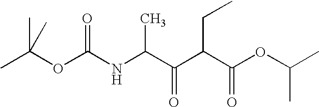

An oven-dried 250 mL round bottom flask was evacuated while hot and allowed to cool under N₂. The flask was twice further evacuated and back-filled with N₂, and charged with anhydrous diisopropylamine (8.50 mL, 60.6 mmol) and anhydrous THF (60 mL). This solution was cooled to 0° C., and n-BuLi (2.5 M solution in hexanes; 24.0 mL, 60.0 mmol) was added dropwise. The resulting solution was allowed to stir at 0° C. for 30 min, and then cooled to −78° C. Isopropyl butyrate (9.10 mL, 60.0 mmol) was added dropwise, and the resulting suspension allowed to stir at −78° C. for 1 h.

A separate, oven-dried 100 mL round bottom flask was evacuated and allowed to cool under N₂. The flask was charged with racemic BOC-alanine (3.41 g, 18.02 mmol) and evacuated and back-filled with N₂. Anhydrous THF (20 mL) was added, and the resulting solution treated with 1,1′-carbonyldiimidazole (3.24 g, 20.0 mmol). Gas evolution occurs immediately. This solution was allowed to stir at room temperature for 30 min, and then added dropwise to the cold suspension of the ester enolate. After an additional hour, the reaction was quenched with glacial AcOH (6.0 mL) and allowed to warm to room temperature. The mixture is partitioned between EtOAc and H₂O, and the aqueous layer was further extracted with EtOAc. The combined organics were washed with brine, dried (MgSO₄), filtered, and concentrated under reduced pressure. The material was purified by silica gel chromatography (R_{f} in 80:20 hexanes:EtOAc=0.18) to give a colorless oil (3.95 g, 73%). ¹H NMR (400 MHz, DMSO d₆) δ 0.77-0.89 (m, 3H) 1.12-1.23 (m, 9H) 1.37 (d, 9 H) 1.63-1.74 (m, 2H) 3.64-3.74 (m, 1H) 4.06-4.17 (m, 1H) 4.88 (dt, 6.28 Hz, 1H) 7.29 (d, 1H). M/Z=301.

### Ethyl(4R)-4-{[(4-chlorophenyl)sulfonyl]amino}-3-oxo-5-phenylpentanoate (starting material 5)

The titled starting material was generated from Starting Material 2a as described below:

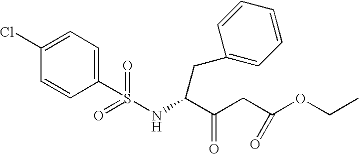

To a suspension of magnesium chloride (1.8 g, 18.9 mmol) in THF (32 mL) was added potassium 3-ethoxy-3-oxopropanoate (4.01 g, 23.5 mmol). The resulting suspension was heated at reflux for 4 h. In another flask, a solution of N-[(4-chlorophenyl)sulfonyl]-D-phenylalanine (Starting Material 2a, 5 g, 14.7 mmol) in THF was cooled to 0° C. and treated with di-1H-imidazol-2-ylmethanone (CDI, 2.63 g, 16.3 mmol). The resulting mixture was warmed to room temperature and transferred to the above prepared magnesium solution via cannular. The solution was stirred overnight. The reaction mixture was poured into a solution of hydrochloride acid (100 mL, 1N) and extracted with EtOAc (3×20 mL). The combined organic layers were dried over Na₂SO₄ and concentrated to yield crude product as a yellow solid, which was purified on silica gel to afford ethyl (4R)-4-{[(4-chlorophenyl)sulfonyl]amino}-3-oxo-5-phenylpentanoate (4.2 g, 70%). M/z 409.

### N²-(tert-Butoxycarbonyl)-N¹-methoxy-N¹-methylalaninamide (Starting Material 6)

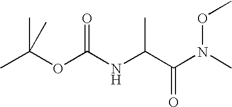

To a solution of Boc-[DL]-Ala-OH (25 g, 132 mmol) and N-methoxyl-N-methylamine hydrochloride salt (19.32 g, 198 mmol) in dry DMF (250 mL) was added DIPEA (117 mL, 673 mmol) under N₂ atm. The resulting solution was stirred for 5 min and treated with HATU (60.2 g, 158.5 mmol). The reaction mixture was stirred for 12 h. Filtration of the reaction mixture gave crude amide that was purified by flash chromatography on silica gel. Yield: 22.1 g (72%). ¹H NMR (300 MHz, CDCl₃) δ: 5.26-5.23 (m, 1H), 4.66-4.63 (m, 1H), 3.7 (s, 3H), 3.13 (s, 3H), 1.41 (s, 9H), 1.29 (d, J=7.4 Hz, 3H). (M+1)/Z=233.1.

1. A compound of formula I 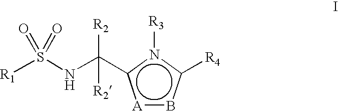
in free or pharmaceutically acceptable salt, prodrug or solvate form, wherein:
A and B are each independently N, NRₐ, O, S, or CR_{b};
Rₐ is H, (C₁-C₆)alkyl, C(O)—(C₁-C₆)alkyl, C(O)—NR′R″, or CO₂(C₁-C₆)alkyl;
R_{b} is H, halo, (C₁-C₆)alkyl, cyano, —C(O)—(C₁-C₆)alkyl, —CO₂(C₁-C₆)alkyl, C(O)—NR′R″, wherein R′ and R″ are each independently at each occurrence H, (C₁-C₆)alkyl or X—R_{c}; —CO₂H, or —SO₂NHR;
R₁ is optionally substituted aryl, heteroaryl, (C₁-C₆)alkyl, aralkyl, heterocycloalkyl, or heteroaralkyl;
R₂ and R_{2′} are each independently H, (C₁-C₆)alkyl, aryl, heteroaryl, aralkyl, or heteroaralkyl, or taken together with the carbon to which they are attached from C═O;
R₃ and R₄ are each independently H, halo, (C₁-C₆)alkyl, (C₃-C₆)cycloalkyl, (C₃-C₆)cycloalkyl(C₁-C₆)alkyl, heterocycloalkyl, aralkyl, aryl, (C₂-C₆)alkenyl, (C₂-C₆)alkynyl, heteroaralkyl, or X—R_{c};
X is S, O, or NR_{d};
R_{c} is H or (C₁-C₆)alkyl;
R_{d} is H, (C₁-C₆)alkyl, aryl, heteroaryl, heterocyclo, (C₂-C₆)alkenyl, (C₂-C₆)alkynyl, aralkyl, heteroaralkyl, (C₃-C₆)cycloalkyl(C₁-C₆)alkyl, heterocycloalkyl(C₁-C₆)alkyl, acyl, acyloxy, acylamino, (C₁-C₆)alkoxycarbonyl(C₁-C₆)alkyl, or cyano; and
each R₁, R₂, R₃, Rₐ, R_{b}, R_{c}, and R_{d} may be optionally substituted on carbon by azido, halo, nitro, cyano, hydroxy, trifluoromethoxy, NR′R″, —CO₂H, C(O)—(C₁-C₆)alkyl, —CO₂(C₁-C₆)alkyl, —C(O)—NR′R″, S(C₁-C₆), SOₚ(C₁-C₆)alkyl, SOₚNH(C₁-C₆)alkyl, SO_{P}NR′R″ (C₂-C₆)alkenyl, (C₂-C₆)alkynyl, or (C₁-C₆)alkoxy, wherein R′ and R″ are each independently hydrogen, (C₁-C₆)alkyl, (C₃-C₆)cycloalkyl, (C₃-C₆)cycloalkyl(C₁-C₆)alkyl, or aryl.

2. The compound according to claim 1 selected from a group consisting of: 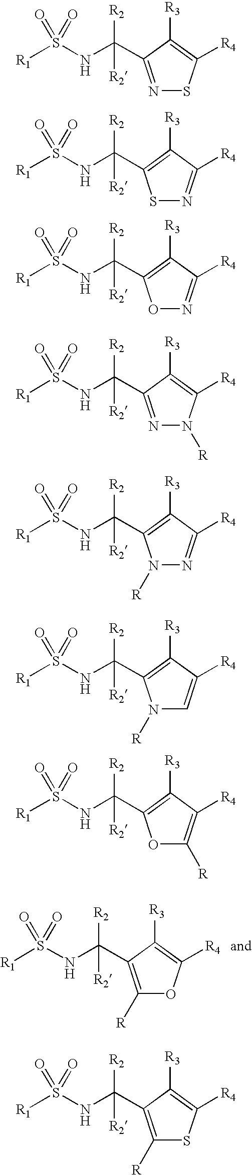
in free or pharmaceutically acceptable salt, prodrug or solvate form, wherein
R₁, R₂, R_{2′}, R₃, and R₄ are as defined for a compound of formula I or II.

3. The compound according to claim 1 selected from a group consisting of: 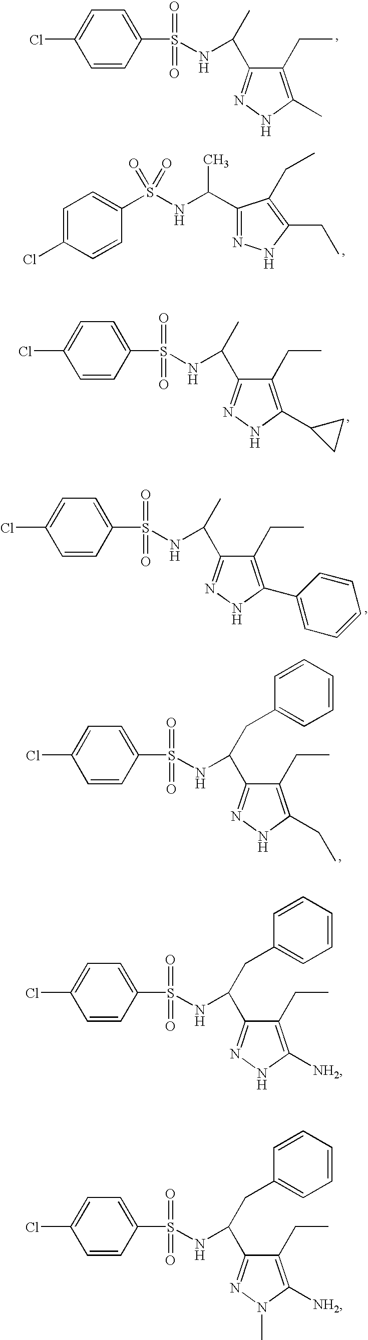 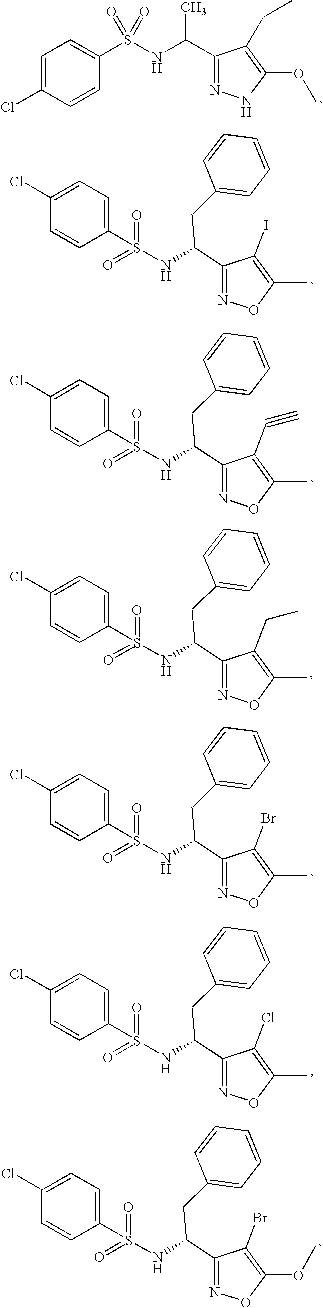 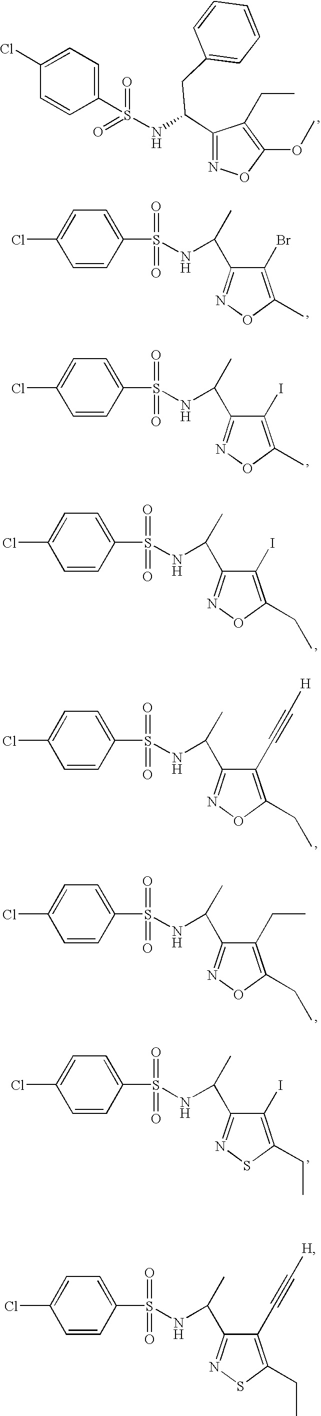 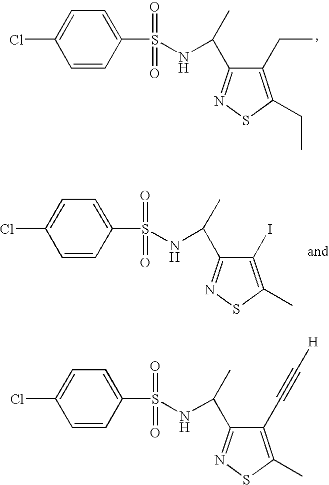
in free or pharmaceutically acceptable salt, prodrug or solvate form.

4. A compound according to claim 1, in free or pharmaceutically acceptable salt, prodrug, or solvate form in association with a pharmaceutically acceptable carrier, diluent, or excipient.

5. (canceled)

6. A method of treating a disease or condition selected from a group consisting of pathologically angiogenic diseases, thrombosis, cardiac infarction, coronary heart diseases, arteriosclerosis, tumors, osteoporosis, inflammations and infections, which method comprises administering to a patient in need of such treatment a compound according to claim 1, in free or pharmaceutically acceptable salt, prodrug, or solvate form.

7. (canceled)

8. A method of treating a disease or condition mediated by Edg-1 which comprises administering to a patient in need of such treatment a compound according claim 1, or a pharmaceutically acceptable salt, prodrug, or solvate form.

9. (canceled)

10. (canceled)

11. (canceled)

12. A pharmaceutical composition comprising a compound according to claim 1, in free or pharmaceutically acceptable salt, prodrug or solvate form, in association with a pharmaceutically acceptable excipient or carrier.

13. A process for the preparation of a compound according to claim 1, in free or pharmaceutically acceptable salt, prodrug or solvate form, which process comprises the step of treating a compound of formula A 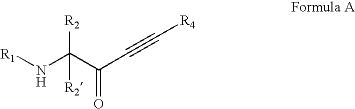
wherein Rₐ, R₁, R₂, R_{2′} and R₄ are as defined according to claim 1; with (i) NH₂OH; (ii) Rₐ—NHNH₂ or (iii) hydroxylamine-O-sulfonic acid and sodium hydrogen sulfide; and optionally halogenating the product thus obtained, and
optionally alkylating the halogenated product thus obtained.

14. A process for the preparation of a compound according to claim 1, in free or pharmaceutically acceptable salt, prodrug or solvate form, which process comprises the step of treating a compound of formula B or C 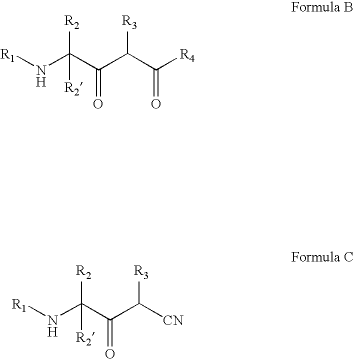
wherein Rₐ, R₁, R₂, R_{2′}, R₃ and R₄ are as defined in claim 1, with RₐNHNH₂.

15. A process for the preparation of a compound according to claim 1, wherein R₄ is OH or C₁₋₆alkoxy, in free or pharmaceutically acceptable salt, prodrug or solvate form, which process comprises the step of treating a compound of formula D 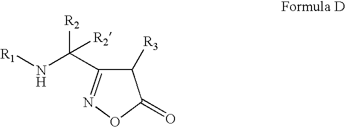
wherein Rₐ, R₁, R₂, R_{2′} and R₃ are as defined in of claim 1 with trimethylsilylmethyl diazane.

16. A process for the preparation of a compound of formula I, wherein R₄ is OH or C₁₋₆alkoxy, in free or pharmaceutically acceptable salt, prodrug or solvate form, which process comprises the step of treating a compound of formula E 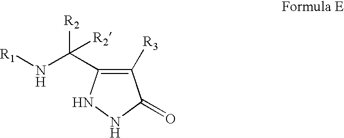
with (i) a base and (ii) haloC₁₋₆alkyl wherein Rₐ, R₁, R₂, R_{2′} and R₃ are as defined in claim 1.

17. A process for the preparation of a compound according to claim 1, wherein R₄ is OH or C₁₋₆alkoxy, in free or pharmaceutically acceptable salt, prodrug or solvate form, which process comprises the step of treating a compound of formula F 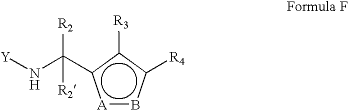
wherein Y is H or a leaving group and R₂, R_{2′}, R₃, R₄, A and B are as defined in claim 1; with R₁—X, wherein X is halo and R₁ is as defined in claim 1; and a base.
